(19) United States
(12) Patent Application Publication
(10) Pub. No.: US 2019/0119759 A1
(43) Pub. Date: Apr. 25, 2019

(54) MUTATIONAL SIGNATURES IN CANCER

(71) Applicant: Genome Research Limited, Hinxton (GB)

(72) Inventors: Serena NIK-ZAINAL, Hinxton (GB); Mike STRATTON, Hinxton (GB); Helen DAVIES, Hinxton (GB); Dominik GLODZIK, Hinxton (GB)

(21) Appl. No.: 16/096,750

(22) PCT Filed: Apr. 28, 2017
(86) PCT No.: PCT/EP2017/060289

§ 371 Date: Oct. 26, 2018

(30) Foreign Application Priority Data
May 1, 2016 (GB) 1607629.1

(51) Int. Cl. C12Q 1/6886; C12Q 1/6809; G16B 40/00

(52) U.S. Cl. CPC C12Q 1/6886; C12Q 1/6809; G16B 40/00; C12Q 2537/165; C12Q 2600/156; C12Q 2535/122; C12Q 2600/106

(57) ABSTRACT

The present invention relates to the identification of a number of mutational signatures in patients with cancer. The mutational signatures include new base substitution signatures and rearrangement signatures. The signatures were identified by whole genome sequencing of 560 breast cancers and the application of new and existing mathematical methods to the base substitution and rearrangements found in those cancers.

### FIELD OF INVENTION

The present invention relates to the identification of a number of mutational signatures in patients with cancer. The mutational signatures include new base substitution signatures and rearrangement signatures. These mutational signatures can be used to characterise the cancer and be used in the identification of treatments. The invention also relates to a method for detecting these signatures.

### BACKGROUND TO THE INVENTION

Somatic mutations are present in all cells of the human body and occur throughout life. They are the consequence of multiple mutational processes, including the intrinsic slight infidelity of the DNA replication machinery, exogenous or endogenous mutagen exposures, enzymatic modification of DNA and defective DNA repair. Different mutational processes generate unique combinations of mutation types, termed “Mutational Signatures”.

In the past few years, large-scale analyses have revealed many mutational signatures across the spectrum of human cancer types.

The mutational theory of cancer proposes that changes in DNA sequence, termed “driver” mutations, confer proliferative advantage upon a cell, leading to outgrowth of a neoplastic clone [1]. Some driver mutations are inherited in the germline, but most arise in somatic cells during the lifetime of the cancer patient, together with many “passenger” mutations not implicated in cancer development [1]. Multiple mutational processes, including endogenous and exogenous mutagen exposures, aberrant DNA editing, replication errors and defective DNA maintenance, are responsible for generating these mutations [10, 12, 13].

Over the past five decades, several waves of technology have advanced the characterisation of mutations in cancer genomes. Karyotype analysis revealed rearranged chromosomes and copy number alterations. Subsequently, loss of heterozygosity analysis, hybridisation of cancer-derived DNA to microarrays and other approaches provided higher resolution insights into copy number changes [14-18]. Recently, DNA sequencing has enabled systematic characterisation of the full repertoire of mutation types including base substitutions, small insertions/deletions, rearrangements and copy number changes [19-23], yielding substantial insights into the mutated cancer genes and mutational processes operative in human cancer.

Mutational processes generating somatic mutations imprint particular patterns of mutations on cancer genomes, termed signatures [10, 28, 30]. Applying a mathematical approach [28] to extract mutational signatures previously revealed five base substitution signatures in breast cancer; signatures 1, 2, 3, 8 and 13 [5, 10].

Germline inactivating mutations in BRCA1 and/or BRCA2 cause an increased risk of early-onset breast [1, 2], ovarian [2, 3], and pancreatic cancer [4], while somatic mutations in these two genes and BRCA1 promoter hypermethylation have also been implicated in development of these cancer types [5, 6]. BRCA1 and BRCA2 are involved in error-free homology-directed double strand break repair [7]. Cancers with defects in BRCA1 and BRCA2 consequently show large numbers of rearrangements and indels due to error-prone repair by non-homologous end joining mechanisms, which assume responsibility for double strand break repair [8, 9].

While defective double strand break repair increases the mutational burden of a cell, thus increasing the chances of acquiring somatic mutations that lead to neoplastic transformation, it also renders a cell more susceptible to cell cycle arrest and subsequent apoptosis when it is exposed to agents such as platinum based antineoplastic drugs [10, 11]. This susceptibility has been successfully leveraged for the development of targeted and less toxic therapeutic strategies for treatment of breast, ovarian, and pancreatic cancers harbouring BRCA1 and/or BRCA2 mutations, notably Poly(ADP-ribose) polymerase (PARP) inhibitors [10, 11]. These treatments cause a multitude of DNA double strand breaks that force neoplastic cells with defective BRCA1 and BRCA2 function into apoptosis since they lack the ability to effectively repair double strand breaks. In contrast, normal cells remain mostly unaffected since their repair machinery is not compromised.

### STATEMENTS OF INVENTION

The present inventors have analysed whole genome sequences of 560 breast cancers to advance understanding of the mutational processes generating somatic mutations. The known mutational signature analysis [28] revealed 7 new base substitution signatures (in addition to the five already known to be present). Of these, five have previously been detected in other cancer types (signatures 5, 6, 17, 18 and 20) whilst two are completely new (signatures 26 and 30).

Similar mathematical principles were extended to genome rearrangements and six completely new “rearrangement signatures” (signatures characterising particular rearrangement mutations) were identified within the 560 breast cancers.

A first aspect of the present invention therefore provides a method of detecting the presence of any one or more of rearrangement signatures 1 to 6 in a DNA sample.

The results described herein suggest that rearrangement signature 3 is strongly associated with BRCA1 mutations or promoter hypermethylation and cancers exhibiting it are thus likely to benefit from either platinum therapy or PARP inhibitors.

The results described herein suggest that rearrangement signature 1 is frequently associated with TP53-mutated, triple-negative breast cancers, showing a high Homologous Recombination Deficiency (HRD) index. Therefore cancers exhibiting this signature are also likely to benefit from either platinum therapy or PARP inhibitors.

The results described herein suggest that rearrangement signature 5 is strongly associated with the presence of BRCA1 mutations or promoter hypermethylation and with BRCA2 mutations. Therefore cancers exhibiting this signature are also likely to benefit from either platinum therapy or PARP inhibitors.

Accordingly, a further aspect of the present invention provides a method of predicting whether a patient with cancer is likely to respond to a PARP inhibitor or a platinum-based drug, the method comprising determining the presence or absence of one or more of rearrangement signatures 1, 3 and/or 5 in a DNA sample obtained from said patient, wherein rearrangement signatures 1, 3 and 5 are defined in Table 1 and a DNA sample is considered to show the presence of a rearrangement signature if the number or proportion of rearrangements in its rearrangement catalogue which are determined to be associated with one of said rearrangement signatures exceeds a predetermined threshold, wherein if one of said rearrangement signatures is present in the sample, the patient is likely to respond to a PARP inhibitor or a platinum-based drug.

In this aspect, and in all of the other aspects of the present invention which relate to the determining the presence of a rearrangement signature, the predetermined threshold may be selected in a number of ways. In particular, different thresholds for this determination may be set depending on the context and the desired certainty of the outcome.

In some embodiments, the threshold will be an absolute number of rearrangements from the rearrangement catalogue of the DNA sample which are determined to be associated with a particular rearrangement signature. If this number is exceeded, then it can be determined that a particular rearrangement signature is present in the DNA sample.

The rearrangement signatures are generally “additive” with respect to each other (i.e. a tumour may be affected by the underlying mutational processes associated with more than one signature and, if this is the case, a sample from that tumour will generally display a higher overall number of rearrangements (being the sum of the separate rearrangements associated with each of the underlying processes), but with the proportion of rearrangements spread over the signatures which are present). As a result, in determining the presence or absence of a particular signature, attention may focus on the absolute number of rearrangements associated with a particular signature in the sample (which may be calculated by the methods described below in other aspects of the invention). Such thresholds are generally better in situations where multiple signatures are present in a sample.

In these embodiments, a signature may be determined to be present if at least 5 and preferably at least 10 informative rearrangements are associated with it.

In other embodiments, the threshold combines the total number of rearrangements detected in the sample (which may be set to ensure that the analysis is representative) along with a proportion of the rearrangements which are associated with a particular signature (again, as determined by the methods described below in other aspects of the invention).

For example, the requirements for determination that a signature is present may be that there are at least 20, preferably at least 40, more preferably at least 50 informative rearrangements and a signature may be deemed to be present if a proportion of at least 10%, preferably at least 20%, more preferably at least 30% of the rearrangements are associated with it. The higher the number of rearrangements present in a sample, the lower the proportional threshold for detection of a specific signature may be.

The proportional thresholds may be adjusted depending on the number of other signatures which make up a significant portion of the rearrangements found in the sample (e.g., if 4 signatures are each present with 20-25% of the rearrangements, then it may be determined that all 4 signatures are present, rather than no signatures at all are present), even if the threshold determined under the present embodiments is 30%.

The above thresholds are based on data obtained from genomes sequenced to 30-40 fold depth. If data is obtained from genomes sequenced at lower coverages, then the number of rearrangements detected overall is likely to be lower, and the thresholds will need to be adjusted accordingly.

In the present aspect, and the other aspects of the invention below which relate to the determination of the presence of any one of rearrangement signatures 1, 3 or 5, the threshold(s) used may be applied to all of these signatures in combination, as well as to each signature individually.

In a further aspect, the invention provides a method of selecting a patient having cancer for treatment with a PARP inhibitor or a platinum-based drug, the method comprising identifying the presence or absence of one or more of rearrangement signatures 1, 3 and/or 5 in a DNA sample obtained from said patient, wherein rearrangement signatures 1, 3 and 5 are defined in Table 1 and a DNA sample is considered to show the presence of a rearrangement signature if the number or proportion of rearrangements in its rearrangement catalogue which are determined to be associated with one or more of said rearrangement signatures each or in combination exceeds a predetermined threshold, and selecting the patient for treatment with a PARP inhibitor or a platinum-based drug if one of said rearrangement signatures is present in the sample.

In a further aspect, the invention provides a PARP inhibitor or a platinum-based drug for use in a method of treatment of cancer in a patient having one or more of rearrangement signatures 1, 3 and/or 5, wherein rearrangement signatures 1, 3 and 5 are defined in Table 1 and a DNA sample is considered to show the presence of a rearrangement signature if the number or proportion of rearrangements in its rearrangement catalogue which are determined to be associated with one or more of said rearrangement signatures each or in combination exceeds a predetermined threshold.

In a further aspect, the invention provides a method of treating cancer in a patient determined to have one or more of rearrangement signatures 1, 3 and/or 5, wherein rearrangement signatures 1, 3 and 5 are defined in Table 1 and a DNA sample is considered to show the presence of a rearrangement signature if the number or proportion of rearrangements in its rearrangement catalogue which are determined to be associated with one or more of said rearrangement signatures each or in combination exceeds a predetermined threshold, the method comprising the step of administering a PARP inhibitor or a platinum-based drug to said patient.

In a further aspect, the invention provides a PARP inhibitor or a platinum-based drug for use in a method of treatment of cancer in a patient, the method comprising:

(i) determining whether one or more of rearrangement signatures 1, 3 and/or 5 is present in a DNA sample obtained from said patient, wherein rearrangement signatures 1, 3 and 5 are defined in Table 1 and a DNA sample is considered to show the presence of a rearrangement signature if the number or proportion of rearrangements in its rearrangement catalogue which are determined to be associated with one or more of said rearrangement signatures each or in combination exceeds a predetermined threshold; and

(ii) administering the PARP inhibitor or a platinum-based drug to a patient if one of said rearrangement signatures is present in said sample.

The methods of the above aspects are to be interpreted as covering the presence of any one of rearrangement signatures 1, 3 or 5 individually within a DNA sample, as well as any combination of those signatures.

The results described herein suggest that rearrangement signature 2 was present in most cancers but was particularly enriched in estrogen-receptor (ER) positive cancers with quiet copy number profiles. Breast cancers that are ER-positive are likely to respond to hormone therapy (e.g. tamoxifen) and therefore breast cancers that are particularly enriched for rearrangement signature 2 are likely to respond to hormone therapy, e.g. treatment with tamoxifen.

In particular examples, the cancer is breast cancer, ovarian cancer or pancreatic cancer.

A further aspect of the present invention provides a method of determining the presence of any one of rearrangement signatures 1 to 6 in a DNA sample obtained from a patient, wherein the rearrangement signatures are defined in Table 1 and a DNA sample is considered to show the presence of a particular rearrangement signature if the number or proportion of rearrangements in its rearrangement catalogue which are determined to be associated with that particular rearrangement signature exceeds a predetermined threshold.

In any of the above aspects and embodiments of the invention, the step of determining or identifying the presence or absence of any of the rearrangement signatures may be as set out in the co-pending application filed on the same day as the present application with application number PCT/EP2017/060279, the contents of which are hereby incorporated by reference. More particularly, the step of determining or identifying the presence or absence of a rearrangement signature may include determining the contributions of known rearrangement signatures to a rearrangement catalogue of a DNA sample by computing the cosine similarity between the rearrangement mutations in said catalogue and the known rearrangement mutational signatures.

Preferably the method includes the further step of, prior to said step of determining, filtering the mutations in said catalogue to remove either residual germline structural variations or known sequencing artefacts or both. Such filtering can be highly advantageous to remove rearrangements from the catalogue which are known to arise from mechanisms other than somatic mutation, and may therefore cloud or obscure the contributions of the rearrangement signatures, or lead to false positive results.

For example, the filtering may use a list of known germline rearrangement or copy number polymorphisms and remove somatic mutations resulting from those polymorphisms from the catalogue prior to determining the contributions of the rearrangement signatures.

As a further example, the filtering may use BAM files of unmatched normal human tissue sequenced by the same process as the DNA sample and discards any somatic mutation which is present in at least two well-mapping reads in at least two of said BAM files. This approach can remove artefacts resulting from the sequencing technology used to obtain the sample.

The classification of the rearrangement mutations may include identifying mutations as being clustered or non-clustered. This may be determined by a piecewise-constant fitting (“PCF”) algorithm which is a method of segmentation of sequential data. In particular embodiments, rearrangements may be identified as being clustered if the average density of rearrangement breakpoints within a segment is a certain factor greater than the whole genome average density of rearrangements for an individual patient's sample. For example the factor may be at least 8 times, preferably at least 9 times and in particular embodiments is 10 times. The inter-rearrangement distance is the distance from a rearrangement breakpoint to the one immediately preceding it in the reference genome. This measurement is already known.

The classification of the rearrangement mutations may include identifying rearrangements as one of: tandem duplications, deletions, inversions or translocations. Such classifications of rearrangement mutations are already known.

The classification of the rearrangement mutations may further include grouping mutations identified as tandem duplications, deletions or inversions by size. For example, the mutations may be grouped into a plurality of size groups by the number of bases in the rearrangement. Preferably the size groups are logarithmically based, for example 1-10 kb, 10-100 kb, 100 kb-1 Mb, 1 Mb-10 Mb and greater than 10 Mb. Translocations cannot be classified by size.

In particular embodiments, in each DNA sample the number of rearrangements Eᵢ associated with the ith mutational signature \vec{S}ᵢ is determined as proportional to the cosine similarity (\vec{C}ᵢ) between the catalogue of this sample \vec{M} and \vec{S}ᵢ:

$${\vec{C}}_{i} = \frac{{\vec{S}}_{i} \cdot \vec{M}}{‖ {\vec{S}}_{i} ‖ ‖ \vec{M} ‖}$$

$$wherein \text{:}$$

$${E}_{i} = \frac{{\vec{C}}_{i}}{{\sum }_{i = 1}^{q} {\vec{C}}_{i}} {\sum }_{j = 1}^{36} {\vec{M}}^{j}$$

wherein \vec{Sᵢ} and \vec{M} are equally-sized vectors with nonnegative components being, respectively, a known rearrangement signature and the mutational catalogue and q is the number of signatures in said plurality of known rearrangement signatures.

The method may further include the step of filtering the number of rearrangements determined to be assigned to each signature by reassigning one or more rearrangements from signatures that are less correlated with the catalogue to signatures that are more correlated with the catalogue. Such filtering can serve to reassign rearrangements from a signature which has only a few rearrangements associated with it (and so is probably not present) to a signature which has a greater number of rearrangement associated with it. This can have the effect of reducing “noise” in the assignment process.

In one embodiment, the step of filtering uses a greedy algorithm to iteratively find an alternative assignment of rearrangements to signatures that improves or does not change the cosine similarity between the catalogue M and the reconstructed catalogue \vec{M}′=S×\vec{E}′ᵢⱼ, wherein \vec{E}′ᵢⱼ is the version of the vector \vec{E} obtained by moving the mutations from the signature i to signature j, wherein, in each iteration, the effects of all possible movements between signatures are estimated, and the filtering step terminates when all of these possible reassignments have a negative impact on the cosine similarity.

In a further aspect, the invention provides a method of detecting mutational signature 26 or mutational signature 30 in a DNA sample, wherein mutational signatures 26 and 30 are defined in Table 2, the method including the steps of: cataloguing the somatic mutations in said sample to produce a mutational catalogue for that sample; determining the contributions of known mutational signatures, including mutational signature 26 or mutational signature 30, to said mutational catalogue by determining a scalar factor for each of a plurality of said known mutational signatures which together minimize a function representing the difference between the mutations in said catalogue and the mutations expected from a combination of said plurality of known mutational signatures scaled by said scalar factors; and if the scalar factor corresponding to mutational signature 26 or mutational signature 30 exceeds a predetermined threshold, identifying said sample as containing corresponding mutational signature 26 or mutational signature 30 respectively.

Preferably the method of this aspect includes the further step of, prior to said step of determining, filtering the mutations in said catalogue to remove either residual germline mutations or known sequencing artefacts or both. Such filtering can be highly advantageous to remove mutations from the catalogue which are known to arise from mechanisms other than somatic mutation, and may therefore cloud or obscure the contributions of the mutational signatures, or lead to false positive results.

For example, the filtering may use a list of known germline polymorphisms and remove somatic mutations resulting from those polymorphisms from the catalogue prior to determining the contributions of the mutational signatures.

As a further example, the filtering may use BAM files of unmatched normal human tissue sequenced by the same process as the DNA sample and discard any somatic mutation which is present in at least two well-mapping reads in at least two of said BAM files. This approach can remove artefacts resulting from the sequencing technology used to obtain the sample.

The method may further include the step of selecting said plurality of known mutational signatures as a subset of all known mutational signatures. By selecting a subset, for example, based on prior knowledge about the sample, the number of possible signatures contributing to the mutational catalogue is reduced, which is likely to increase the accuracy of the determining step.

For example, the subset of mutational signatures may be selected based on biological knowledge about the DNA sample or the mutational signatures or both. Thus, it may be immediately apparent that a certain DNA sample cannot have resulted from a particular mutational signature as a result of characteristics of the DNA sample and the particular mutational signature. Further possibilities are described in more detail in the embodiments below.

In particular embodiments, the step of determining may determine the scalars Eᵢ which minimize the Frobenius norm:

$$min {‖ \vec{M} - {\sum }_{i = 1}^{q} ( \vec{{S}_{i}} \times {E}_{i} ) ‖}_{2}^{F}$$

wherein \vec{Sᵢ} and \vec{M} are equally-sized vectors with nonnegative components being, respectively, a consensus mutational signature and the mutational catalogue and q is the number of signatures in said plurality of known mutational signatures, and wherein Eᵢ are further constrained by the requirements that 0≤Eᵢ≤∥\vec{Sᵢ}∥₁, i=1 . . . q, and

$${\sum }_{i = 1}^{q} {E}_{i} = {‖ \vec{{S}_{i}} ‖}_{1} .$$

### BRIEF DESCRIPTION OF THE FIGURES & TABLES

FIG. 1 summarises the cohort of 560 breast cancer genomes that were studied by the inventors;

FIG. 2 is a diagram showing seven major subgroups exhibiting distinct associations with other genomic, histological or gene expression features, along with the six rearrangement signatures extracted from the data.

FIG. 3 is a further summary of the cohort of genomes that were studied;

FIG. 4 shows the base substitution signatures that were identified in the cohort;

FIG. 5 shows the rearrangement signatures that were identified in the cohort;

FIG. 6 shows the clinical relevance of the clustering based on the identified rearrangement signatures;

FIG. 7 shows the breakpoint characteristics in which bars to the left of “blunt” are non-template sequence, the bar labelled “blunt” is blunt end-joining and the bars to the right of “blunt” are microhomology; and

FIG. 8 is a flow chart showing the outline steps in a method of determining the presence of a rearrangement signature according to an embodiment of the present invention.

Table 1 shows a quantitative definition of a number of rearrangement signatures; and Table 2 shows a quantitative definition of base substitution signatures 26 and 30.

### DETAILED DESCRIPTION

The present invention is based on the finding that subset of patients with cancers have a particular mutational or rearrangement signatures. The rearrangement signatures are defined in more detail below and are set out quantitatively in Table 1. The mutational (or “base-substitution”) signatures are set out quantitatively in Table 2.

As identified further below, several of the rearrangement signatures (signatures 1, 3 and 5) are associated with failure of double-stranded break repair by homologous recombination and/or lack BRCA1/2 defects and therefore, cancer patients having one or more of these rearrangement signatures are likely to benefit from either platinum therapy or treatment with PARP inhibitors.

The invention therefore relates, inter alia, to a method of predicting whether a patient with cancer is likely to respond to a PARP inhibitor or a platinum-based drug or to a method of selecting a patient having cancer for treatment with a PARP inhibitor or a platinum-based drug based on the presence or absence of one or more of rearrangement signatures 1, 3 or 5 in a DNA sample obtained from said patient.

It is noted that the phrase “presence of one or more of rearrangement signatures 1, 3 or 5” as used herein includes, inter alia, the presence of any one of those signatures, as well as the presence of any combination of those signatures. In particular, it includes the presence of all three of these signatures even if, due to the presence of all of these signatures, the proportion of rearrangements in the DNA sample which are determined to be associated with any one of those signatures is lower than might be otherwise be considered appropriate to reach a determination that a particular signature is present.

The patient is preferably a human patient.

Cancer patients having rearrangement signatures 1, 3 and/or 5 are likely to have a failure of DNA double strand repair by homologous recombination and to be susceptible to drugs that generate double strand breaks, e.g. a PARP inhibitor or a platinum-based drug.

The enzyme poly ADP ribose polymerase (PARP1) is a protein that is important for repairing single-strand breaks, also known as ‘nicks’. If such nicks persist unrepaired until DNA is replicated then the replication itself can cause formation of multitude of double strand breaks. Drugs that inhibit PARP1 cause large amounts of double strand breaks. In tumours with failure of double-strand DNA break repair by error-free homologous recombination, the inhibition of PARP1 results in inability to repair these double strand breaks and leads to the death of the tumour cells. The PARP inhibitor for use in the present invention is preferably a PARP1 inhibitor. Examples of PARP inhibitors include: Iniparib, Talazoparib, Olaparib, Rucaparib, and Veliparib.

Platinum-based antineoplastic drugs are chemotherapeutic agents used to treat cancer. They are coordination complexes of platinum that cause crosslinking of DNA as monoadduct, interstrand crosslinks, intrastrand crosslinks or DNA protein crosslinks. Mostly they act on the adjacent N-7 position of guanine, forming 1, 2 intrastrand crosslink. The resultant crosslinking inhibits DNA repair and/or DNA synthesis in cancer cells. Some commonly used platinum-based antineoplastic drugs include: cisplatin, carboplatin, oxaliplatin, satraplatin, picoplatin, Nedaplatin, Triplatin, and Lipoplatin.

The presence or absence of rearrangement signatures 1, 3 and/or 5 is determined in DNA samples obtained from the patient. Preferably, these are whole genome samples and the presence or absence of the rearrangement signature(s) may be determined by whole genome sequencing. The DNA samples may be whole-exome samples and the presence or absence of the rearrangement signature(s) may be determined by whole exome sequencing.

Exome sequencing is a technique for sequencing all the protein-coding genes in a genome (known as the exome). It consists of first selecting only the subset of DNA that encodes proteins (known as exons), and then sequencing that DNA using any high throughput DNA sequencing technology. There are 180,000 exons, which constitute about 1% of the human genome, or approximately 30 million base pairs.

The DNA samples are preferably obtained from both tumour and normal tissues obtained from the patient, e.g. blood sample from the patient and tumour tissue obtained by a biopsy. Somatic mutations in the tumour sample are detected, standardly, by comparing its genomic sequences with the one of the normal tissue.

The invention also relates to the treatment of cancer with a PARP inhibitor or a platinum-based drug in a patient having one or more of rearrangement signatures 1, 3 and/or 5.

For example, the PARP inhibitor or platinum-based drug may be for use in a method of treatment of cancer in a patient having one or more of rearrangement signatures 1, 3 and/or 5. Prior to treatment, the method may comprise the step of determining whether one or more of these rearrangement signatures is present in DNA samples obtained from said patient. Preferably, these are whole genome samples and the presence or absence of the rearrangement signature(s) may be determined by whole genome sequencing. The DNA samples may be whole-exome samples and the presence or absence of the rearrangement signature(s) may be determined by whole exome sequencing.

The DNA samples are preferably obtained from both tumour and normal tissues obtained from the patient, e.g. blood sample from the patient and tumour tissue obtained by a biopsy. Somatic mutations in the tumour sample are detected, standardly, by comparing its genomic sequences with the one of the normal tissue.

The method of treatment comprises the step of administering the PARP inhibitor or platinum-based drug to a cancer patient having one or more of rearrangement signatures 1, 3 and/or 5. Any suitable route of administration may be used.

The patient to be treated is preferably a human patient.

The invention also relates to a method for detecting any one of rearrangement signatures 1-6 or mutational signatures 26 and 30 in a DNA sample obtained from a subject. This method is applicable to any subject, including a subject with breast, ovarian, pancreatic or gastric cancer. Further details of such methods are set out below.

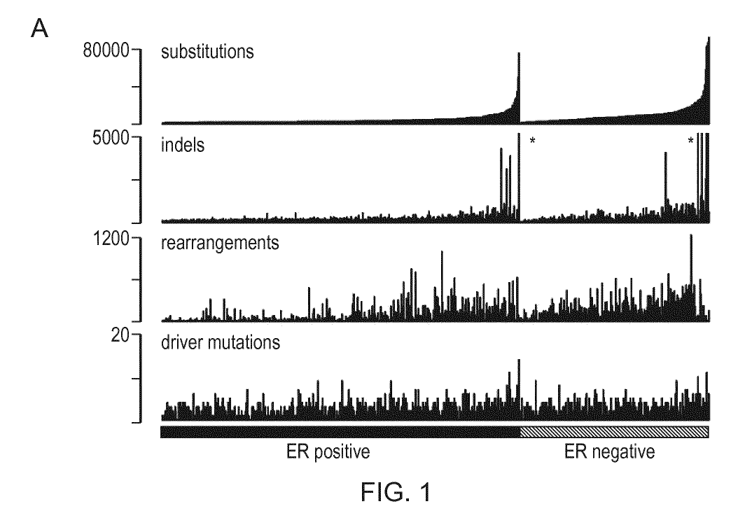

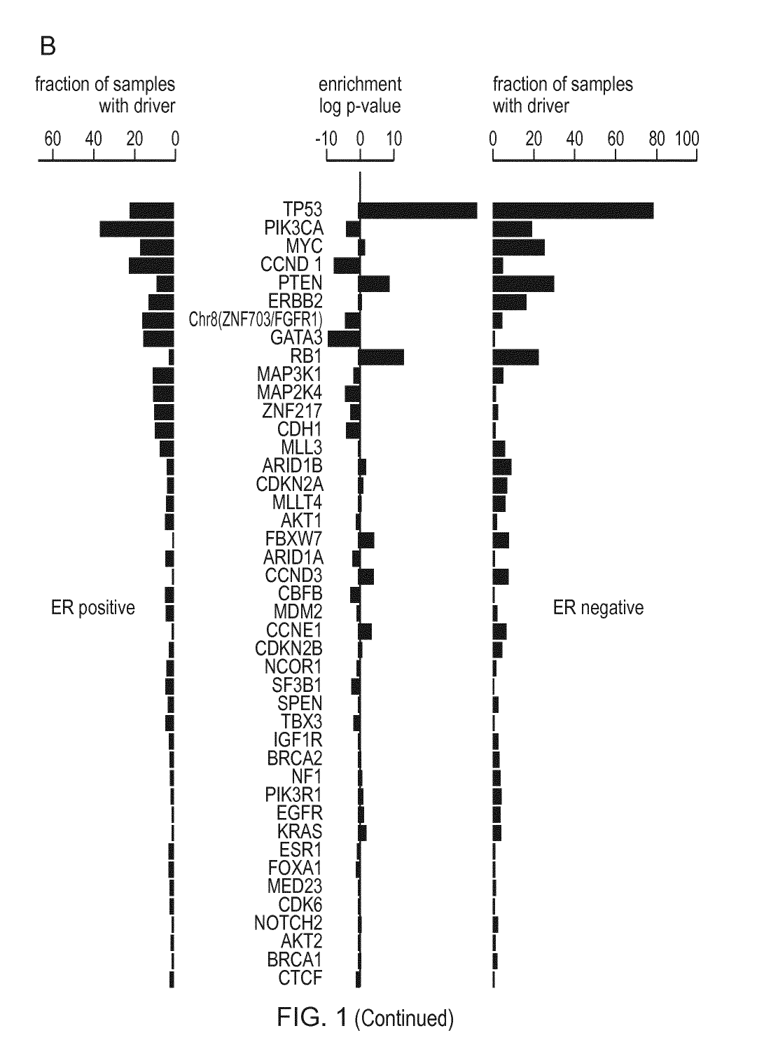

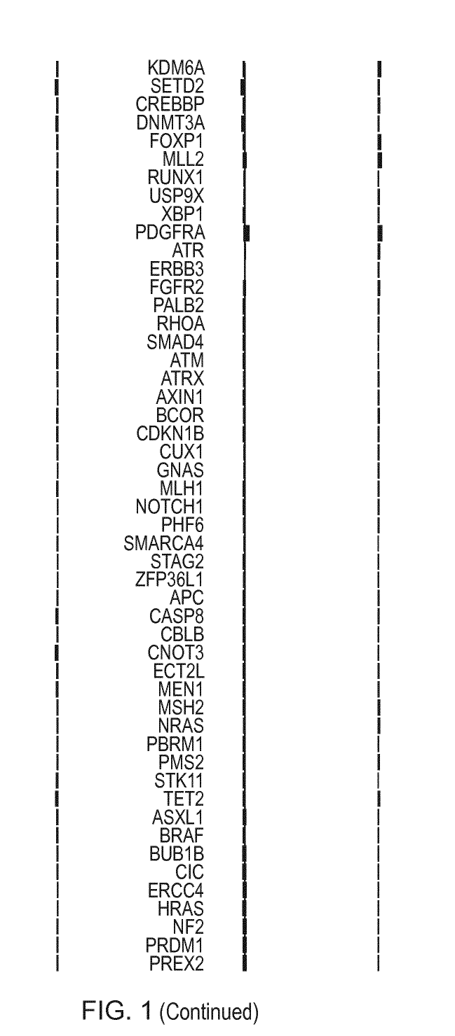

Identification Of Rearrangement Signatures Linked to Cancer The complete genomes of 560 breast cancers and non-neoplastic tissue from each individual (556 female and four male) were sequenced (FIG. 1A). 3,479,652 somatic base substitutions, 371,993 small indels and 77,695 rearrangements were detected, with substantial variation in the number of each between individual samples (FIG. 1B). Transcriptome sequence, microRNA expression, array based copy number and DNA methylation data were obtained from subsets of cases.

To enable investigation of signatures of rearrangement mutational processes, a rearrangement classification was adopted incorporating 32 subclasses.

In many cancer genomes, large numbers of rearrangements are regionally clustered, for example in zones of gene amplification. Therefore, the rearrangements were first classified into those that occurred as clusters or were dispersed, further sub-classified into deletions, inversions and tandem duplications, and then according to the size of the rearranged segment. The final category in both groups was inter-chromosomal translocations.

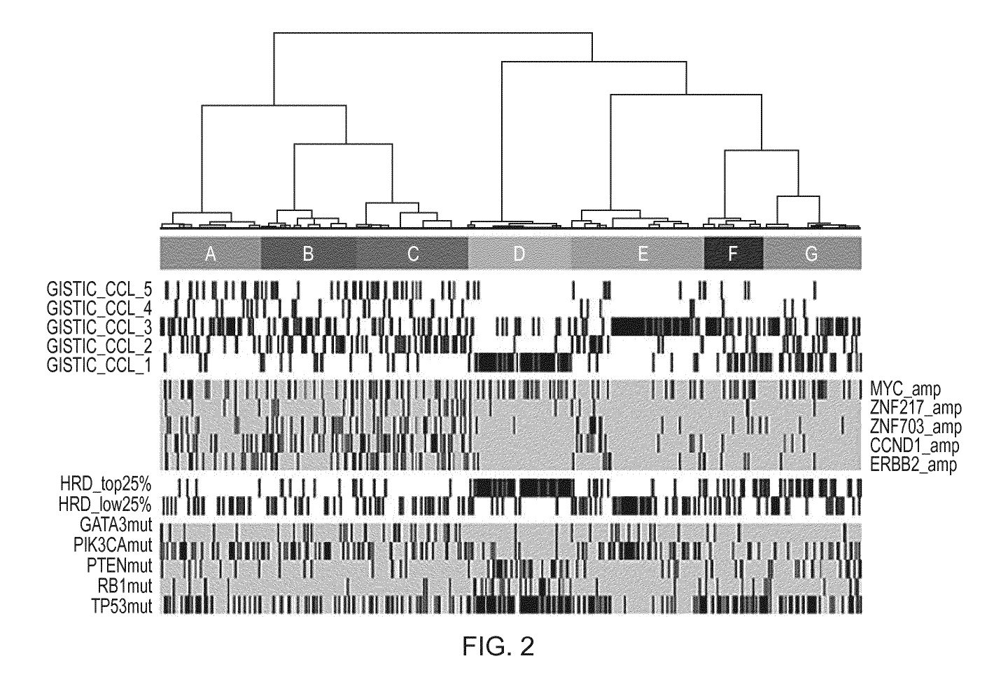

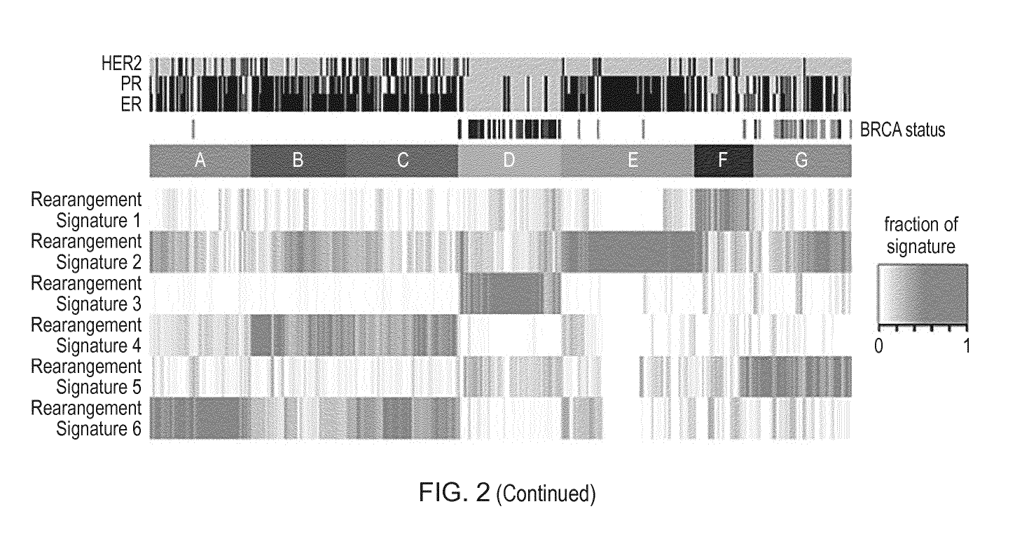

Application of the mathematical framework used for base substitution signatures [5, 10, 28] extracted six rearrangement signatures. Unsupervised hierarchical clustering on the basis of the proportion of rearrangements attributed to each signature in each breast cancer yielded seven major subgroups exhibiting distinct associations with other genomic, histological or gene expression features as shown in FIG. 2.

Rearrangement Signature 1 (9% of all rearrangements) and Rearrangement Signature 3 (18% rearrangements) were characterised predominantly by tandem duplications. Tandem duplications associated with Rearrangement Signature 1 were mostly >100 kb, and those with Rearrangement Signature 3 <10 kb. More than 95% of Rearrangement Signature 3 tandem duplications were concentrated in 15% of cancers (FIG. 2, Cluster D), many with several hundred rearrangements of this type. Almost all cancers (91%) with BRCA1 mutations or promoter hypermethylation were in this group, which was enriched for basal-like, triple negative cancers and copy number classification of a high Homologous Recombination Deficiency (HRD) index [31-33]. Thus, inactivation of BRCA1, but not BRCA2, may be responsible for the Rearrangement Signature 3 small tandem duplication mutator phenotype.

Accordingly the presence or absence of Rearrangement Signature 3, particularly, but not exclusively, in comparison to the presence or absence of Rearrangement Signatures 1 and 5 may be used to distinguish between cancers which have inactivation of BRCA1 but not BRCA2.

More than 35% of Rearrangement Signature 1 tandem duplications were found in just 8.5% of the breast cancers and some cases had hundreds of these (FIG. 2, Cluster F). The cause of this large tandem duplication mutator phenotype is unknown. Cancers exhibiting it are frequently TP53-mutated, relatively late diagnosis, triple-negative breast cancers, showing enrichment for base substitution signature 3 and a high Homologous Recombination Deficiency (HRD) index (FIG. 2) but do not have BRCA1/2 mutations or BRCA1 promoter hypermethylation.

Rearrangement Signature 1 and 3 tandem duplications were generally evenly distributed over the genome. However, there were nine locations at which recurrence of tandem duplications was found across the breast cancers and which often showed multiple, nested tandem duplications in individual cases. These may be mutational hotspots specific for these tandem duplication mutational processes although we cannot exclude the possibility that they represent driver events.

Rearrangement Signature 5 (accounting for 14% rearrangements) was characterised by deletions <100 kb. It was strongly associated with the presence of BRCA1 mutations or promoter hypermethylation (FIG. 2, Cluster D), BRCA2 mutations (FIG. 2, Cluster G) and with Rearrangement Signature 1 large tandem duplications (FIG. 2, Cluster F).

Rearrangement Signature 2 (accounting for 22% rearrangements) was characterised by non-clustered deletions (>100 kb), inversions and interchromosomal translocations, was present in most cancers but was particularly enriched in ER positive cancers with quiet copy number profiles (FIG. 2, Cluster E, GISTIC Cluster 3). Rearrangement Signature 4 (accounting for 18% of rearrangements) was characterised by clustered interchromosomal translocations while Rearrangement Signature 6 (19% of rearrangements) by clustered inversions and deletions (FIG. 2, Clusters A, B & C).

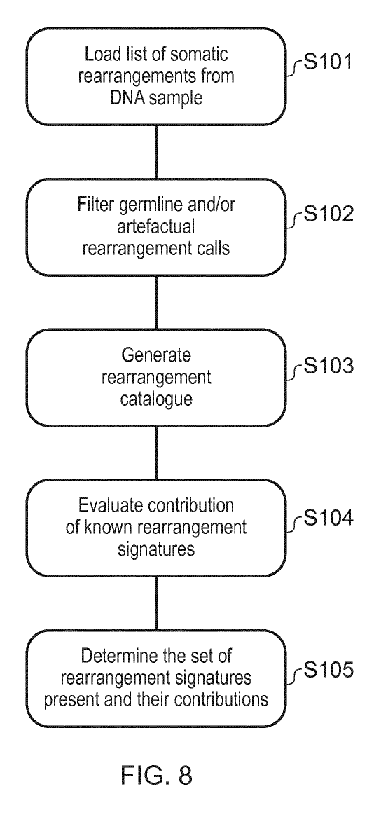

Short segments (1-5 bp) of overlapping microhomology characteristic of alternative methods of end joining repair were found at most rearrangements [10, 24]. Rearrangement Signatures 2, 4 and 6 were characterised by a peak at 1 bp of microhomology while Rearrangement Signatures 1, 3 and 5, associated with homologous recombination DNA repair deficiency, exhibited a peak at 2 bp (FIG. 8). Thus, different end-joining mechanisms may operate with different rearrangement processes. A proportion of breast cancers showed Rearrangement Signature 5 deletions with longer (>10 bp) microhomologies involving sequences from short-interspersed nuclear elements (SINEs), most commonly AluS (63%) and AluY (15%) family repeats (FIG. 8). Long segments (more than 10 bp) of non-templated sequence were particularly enriched amongst clustered rearrangements.

Methods

Sample Selection

DNA was extracted from 560 breast cancers and normal tissue (peripheral blood lymphocytes, adjacent normal breast tissue or skin). Samples were subjected to pathology review and only samples assessed as being composed of >70% tumor cells, were accepted for inclusion in the study.

Massively-Parallel Sequencing and Alignment

Short insert 500 bp genomic libraries were constructed, flowcells prepared and sequencing clusters generated according to Illumina library protocols [34]. 108 base/100 base (genomic) paired-end sequencing were performed on Illumina GAllx, Hiseq 2000 or Hiseq 2500 genome analyzers in accordance with the Illumina Genome Analyzer operating manual. The average sequence coverage was 40.4 fold for tumour samples and 30.2 fold for normal samples.

Short insert paired-end reads were aligned to the reference human genome (GRCh37) using Burrows-Wheeler Aligner, BWA (v0.5.9) [35].

Processing of Genomic Data

CaVEMan (Cancer Variants Through Expectation Maximization: http://cancerit.github.io/CaVEMan/) was used for calling somatic substitutions. Indels in the tumor and normal genomes were called using a modified Pindel version 2.0. (http://cancerit.github.io/cgpPindel/) on the NCBI37 genome build [36].

Structural variants were discovered using a bespoke algorithm, BRASS (BReakpoint AnalySiS) (https://github.com/cancerit/BRASS) through discordantly mapping paired-end reads. Next, discordantly mapping read pairs that were likely to span breakpoints, as well as a selection of nearby properly-paired reads, were grouped for each region of interest. Using the Velvet de novo assembler [37], reads were locally assembled within each of these regions to produce a contiguous consensus sequence of each region. Rearrangements, represented by reads from the rearranged derivative as well as the corresponding non-rearranged allele were instantly recognisable from a particular pattern of five vertices in the de Bruijn graph (a mathematical method used in de novo assembly of (short) read sequences) of component of Velvet. Exact coordinates and features of junction sequence (e.g. microhomology or non-templated sequence) were derived from this, following aligning to the reference genome, as though they were split reads.

Annotation was according to ENSEMBL version 58.

Single nucleotide polymorphism (SNP) array hybridization using the Affymetrix SNP6.0 platform was performed according to Affymetrix protocols. Allele-specific copy number analysis of tumors was performed using ASCAT (v2.1.1), to generate integral allele-specific copy number profiles for the tumor cells [38]. ASCAT was also applied to NGS data directly with highly comparable results.

12.5% of the breast cancers were sampled for validation of substitutions, indels and/or rearrangements in order to make an assessment of the positive predictive value of mutation-calling.

Mutational Signatures Analysis

Mutational signatures analysis was performed following a three-step process: (i) hierarchical de novo extraction based on somatic substitutions and their immediate sequence context, (ii) updating the set of consensus signatures using the mutational signatures extracted from breast cancer genomes, and (iii) evaluating the contributions of each of the updated consensus signatures in each of the breast cancer samples. These three steps are discussed in more detail in the next sections.

Hierarchical De Novo Extraction of Mutational Signatures

The mutational catalogues of the 560 breast cancer whole genomes were analysed for mutational signatures using a hierarchical version of the Wellcome Trust Sanger Institute mutational signatures framework [28]. Briefly, all mutation data was converted into a matrix, M that is made up of 96 features comprising mutations counts for each mutation type (C>A, C>G, C>T, T>A, T>C, and T>G; all substitutions are referred to by the pyrimidine of the mutated Watson-Crick base pair) using each possible 5′ (C, A, G, and T) and 3′ (C, A, G, and T) context for all samples. After conversion, the previously developed algorithm was applied in a hierarchical manner to the matrix M that contains K mutation types and G samples. The algorithm deciphers the minimal set of mutational signatures that optimally explains the proportion of each mutation type and then estimates the contribution of each signature across the samples. More specifically, the algorithm makes use of a well-known blind source separation technique, termed nonnegative matrix factorization (NMF). NMF identifies the matrix of mutational signature, P and the matrix of the exposures of these signatures, E, by minimizing a Frobenius norm while maintaining non-negativity:

$${‖ M - P \times E ‖}_{F}^{2}$$

The method for deciphering mutational signatures, including evaluation with simulated data and list of limitations, can be found in [29]. The framework was applied in a hierarchical manner to increase its ability to find mutational signatures present in few samples as well as mutational signatures exhibiting a low mutational burden. More specifically, after application to the original matrix M containing 560 samples, we evaluated the accuracy of explaining the mutational patterns of each of the 560 breast cancers with the extracted mutational signatures. All samples that were well explained by the extracted mutational signatures were removed and the framework was applied to the remaining sub-matrix of M. This procedure was repeated until the extraction process did not reveal any new mutational signatures. Overall, the approach extracted 12 unique mutational signatures operative across the 560 breast cancers

Updating the Set of Consensus Mutational Signatures

The 12 hierarchically extracted breast cancer signatures were compared to the census of consensus mutational signatures [28]. 11 of the 12 signatures closely resembled previously identified mutational patterns. The patterns of these 11 signatures, weighted by the numbers of mutations contributed by each signature in the breast cancer data, were used to update the set of consensus mutational signatures as previously done in [28]. 1 of the 12 extracted signatures is novel and at present, unique for breast cancer. This novel signature is consensus signature 30 (http://cancersangerac.uk/cosmic/signatures).

Evaluating the Contributions of Consensus Mutational Signatures in 560 Breast Cancers

The complete compendium of consensus mutational signatures that was found in breast cancer includes: signatures 1, 2, 3, 5, 6, 8, 13, 17, 18, 20, 26, and 30. The presence of all these signatures in the 560 breast cancer genomes was evaluated by re-introducing them into each sample. More specifically, the updated set of consensus mutational signatures was used to minimize the constrained linear function for each sample:

$${min}_{{Exposures}_{i} \geq 0} {‖ SampleMutations - {\sum }_{i = 1}^{N} ( \vec{{Signature}_{l}} * {Exposure}_{i} ) ‖}_{F}^{2}$$

Here, \vec{Signatureᵢ} represents a vector with 96 components (corresponding to a consensus mutational signature with its six somatic substitutions and their immediate sequencing context) and Exposure is a nonnegative scalar reflecting the number of mutations contributed by this signature. N is equal to 12 and it reflects the number of all possible signatures that can be found in a single breast cancer sample. Mutational signatures that did not contribute large numbers (or proportions) of mutations or that did not significantly improve the correlation between the original mutational pattern of the sample and the one generated by the mutational signatures were excluded from the sample. This procedure reduced over-fitting the data and allowed only the essential mutational signatures to be present in each sample.

Rearrangement Signatures

Clustered Vs Non-Clustered Rearrangements

The inventors sought to separate rearrangements that occurred as focal catastrophic events or focal driver amplicons from genome-wide rearrangement mutagenesis using a piecewise constant fitting (PCF) method. For each sample, both breakpoints of each rearrangement were considered individually and all breakpoints were ordered by chromosomal position. The inter-rearrangement distance, defined as the number of base pairs from one rearrangement breakpoint to the one immediately preceding it in the reference genome, was calculated. Putative regions of clustered rearrangements were identified as having an average inter-rearrangement distance that was at least 10 times greater than the whole genome average for the individual sample. PCF parameters used were γ=25 and kmin=10. The respective partner breakpoint of all breakpoints involved in a clustered region are likely to have arisen at the same mechanistic instant and so were considered as being involved in the cluster even if located at a distant chromosomal site.

Classification—Types and Size

In both classes of rearrangements, clustered and non-clustered, rearrangements were subclassified into deletions, inversions and tandem duplications, and then further subclassified according to size of the rearranged segment (1-10 kb, 10 kb-100 kb, 100 kb-1 Mb, 1 Mb-10 Mb, more than 10 Mb). The final category in both groups was interchromosomal translocations.

Rearrangement Signatures by NNMF

The classification produces a matrix of 32 distinct categories of structural variants across 544 breast cancer genomes. This matrix was decomposed using the previously developed approach for deciphering mutational signatures by searching for the optimal number of mutational signatures that best explains the data without over-fitting the data [28].

The methods according to embodiments of the invention set out below determine the presence or absence of a rearrangement signature or a base-substitution signature in DNA samples obtained from a single patient. Preferably, these are whole genome samples and the presence or absence of mutational signatures may be determined by whole genome sequencing. The DNA samples may be whole-exome samples and the presence or absence of mutational signatures may be determined by whole exome sequencing. Exome sequencing is a technique for sequencing all the protein-coding genes in a genome (known as the exome). It consists of first selecting only the subset of DNA that encodes proteins (known as exons), and then sequencing that DNA using any high throughput DNA sequencing technology. There are 180,000 exons, which constitute about 1% of the human genome, or approximately 30 million base pairs.

The DNA samples are preferably obtained from both tumour and normal tissues obtained from the patient, e.g. blood sample from the patient and breast tumour tissue obtained by a biopsy. Somatic mutations in the tumour sample are detected, standardly, by comparing its genomic sequences with the one of the normal tissue.

Method of Detection of Rearrangement Signatures in a Single Patient

In embodiments of the present invention, detection of a rearrangement signature in the DNA obtained from a single patient is performed. In these embodiments, this detection is performed by a computer-implemented method or tool that examines a list of somatic mutations generated through high-coverage or low-pass sequencing of nucleic acid material obtained from fresh-frozen derived DNA, circulating tumour DNA of formalin-fixed paraffin-embedded (FFPE) DNA representative of a suspected or known tumour from a patient. The steps of this method are illustrated schematically in FIG. 1.

The list of somatic mutations for these embodiments can be provided in variety of different formats (including, VCF, BEDPE, text etc.) but at the very minimum needs to contain the following information: genome assembly version, lower breakpoint chromosome, lower breakpoint coordinate, higher breakpoint chromosome, higher breakpoint coordinate and either rearrangement class (inversion, tandem duplication deletion, translocation) or strand information of lower and higher breakpoints to enable orientation of rearrangement breakpoints in order to correctly classify them.

In broad terms, after loading the list of somatic mutations from the DNA sample (S101) the tool firstly filters out any known germline and/or artifactual somatic mutations (S102), then generates the rearrangement catalogue of the sample, then classifies the rearrangements based on the classification described below (S103), then evaluates the contributions of known consensus rearrangement mutational signatures to this sample (S104) and finally determines the set of signatures of rearrangement processes, and their respective contributions, that are operative in the sample (S105).

By default, the patterns of the consensus rearrangement signatures are those shown in Table 1, but these patterns of mutational signatures could be also user provided and the method is not limited to known signatures and can be readily applied to new or modified signatures which are discovered in the future.

Filtering Initial Data

Prior to analysing the data, the input list of somatic rearrangements is extensively filtered to remove any residual germline mutations as well as technology specific sequencing artefacts.

Germline rearrangements or copy number polymorphisms are filtered out from the lists of reported somatic mutations using the complete list of germline mutations from dbSNP [25], 1000 genomes project [26], NHLBI GO Exome Sequencing Project [27] and 69 Complete Genomics panel (http://www.completegenomics.com/public-data/69-Genomes/).

Technology specific sequencing artefacts (related to library-marking or sequencing chemistry) and mapping-related artefacts caused by errors or biases in the reference genome, are filtered out by using panels of BAM files of unmatched normal human tissues containing at least 100 normal whole-genomes. The remaining somatic mutations are used to construct the mutational catalogue of the examined sample.

Generating the Mutational Catalogue for a Sample

The list of remaining (i.e., post-filtered) somatic rearrangements is used to generate the rearrangement mutational catalogue of a sample.

(1) Clustered Vs Non-Clustered

The first classification applied to the mutations is whether they are clustered (closely-grouped) or not.

To distinguish collections of rearrangements that are clustered or close together in a patient's cancer genome from other rearrangements that are distributed or dispersed throughout the genome, the data is parsed through a PCF-based algorithm. The PCF (Piecewise-Constant-Fitting) algorithm is a method of segmentation of sequential data.

Before applying PCF, a number of steps are performed on the rearrangement data.

Unlike substitutions or indels that have a single genomic coordinate to signify their position, rearrangements have two coordinates or “breakpoints” that identify two distant genomic loci that have been brought together by a large structural mutation event.

First, both breakpoints of each rearrangement are treated independently. The breakpoints are then sorted according to reference genomic coordinate in each sample. The intermutation distance (IMD), defined as the number of base pairs from one rearrangement breakpoint to the one immediately preceding it in the reference genome, is calculated for each breakpoint. The calculated IMD is then fed to the PCF algorithm.

To identify regions of “clustered” rearrangements from “non-clustered” rearrangements, a set of rearrangements was required to have an average density of rearrangement breakpoints that was at least 10 times greater than the whole genome average density of rearrangements for an individual patient's sample. Additionally, a gamma parameter (a measure of smoothness of segmentation) was stipulated, γ=25, and required that a minimum of 10 breakpoints were present in each region, before it could be classified as a cluster of rearrangements. Biologically, the respective partner breakpoint of any rearrangement involved in a clustered region is likely to have arisen at the same mechanistic instant and so can be considered as being involved in the cluster even if located at a distant genomic site according to the reference genome.

Thus rearrangements are first classified as “clustered” or “non-clustered.

(2) Type and Size

In both clustered and non-clustered categories, rearrangements are then classified based on the information provided into the main classes of rearrangements:
tandem duplications
deletions
inversions
translocations

Tandem duplications, deletions and inversions can then be categorised into the following 5 size groups where the size of a rearrangement is obtained through subtracting the lower breakpoint coordinate from the higher one.
1-10 kb
10-100 kb
100 kb-1 Mb
1 Mb-10 Mb
>10 Mb

Translocations are the exception and cannot be classified by size.

In all, there will be 16 subgroups of clustered and 16 subgroups of non-clustered rearrangements and thus 32 categories altogether. These are listed in Table 1.

The outcome of this classification can then be fed into a latent variable analysis such as NNMF, to obtain a non-negative vector of 32 elements describing each rearrangement signature.

Evaluating the Numbers of Somatic Mutations Attributed to Re-Arrangement Signatures in the Mutational Catalogue of the Examined Sample

Calculating the contributions of all mutational signatures is performed by estimating the number of mutations associated to the consensus patterns of the signatures of all operative mutational processes in the sample. Below a method of estimating this using non-negative matrix factorisation (NNMF) is set out, although alternative methods such as EMU or a hierarchical Dirichlet process (HDP) may equally be used.

More specifically, all consensus rearrangement signatures are examined as a set P containing s vectors

$$P = \{ [ \begin{matrix}{p}_{1}^{1} \\ ⋮ \\ {p}_{1}^{32}\end{matrix} ] , [ \begin{matrix}{p}_{2}^{1} \\ ⋮ \\ {p}_{2}^{32}\end{matrix} ] … [ \begin{matrix}{p}_{s - 1}^{1} \\ ⋮ \\ {p}_{s - 1}^{32}\end{matrix} ] , [ \begin{matrix}{p}_{s}^{1} \\ ⋮ \\ {p}_{s}^{32}\end{matrix} ] \} ,$$

where each of the vectors is a discrete probability density function reflecting a consensus rearrangement signature. For the currently known rearrangement signatures, these vectors are set out in the respective columns of Table 1. Here, s refers to the number of known consensus rearrangement signatures (currently 6) and the 32 nonnegative components of each vector correspond to the different categories of rearrangements (i.e., clustered/non-clustered, type & size) of these consensus rearrangement signatures.

The contributions of all consensus rearrangement signatures are estimated independently for the mutational catalogue of the examined sample. The estimation algorithm consists of computing the cosine similarity between each signature and examined sample. For a set of vectors S_{1 . . . q}, q≤s, the cosine similarity \vec{C}ᵢ is given by:

$${\vec{C}}_{i} = \frac{{\vec{S}}_{i} \cdot \vec{M}}{‖ {\vec{S}}_{i} ‖ ‖ \vec{M} ‖}$$

The number of rearrangements Eᵢ associated with the ith mutational signature \vec{S}ᵢ is proportional to the cosine similarity (\vec{C}ᵢ):

$${E}_{i} = \frac{{\vec{C}}_{i}}{{\sum }_{i = 1}^{q} {\vec{C}}_{i}} {\sum }_{j = 1}^{36} {\vec{M}}^{j}$$

wherein \vec{Sᵢ} and \vec{M} are equally-sized vectors with nonnegative components being, respectively, a known rearrangement signature and the mutational catalogue and q is the number of signatures in said plurality of known rearrangement signatures.

In the above equation, \vec{Sᵢ} and \vec{M} represent vectors with 32 nonnegative components (corresponding to the clustered/non-clustered characteristic and the type and size of the rearrangements) reflecting, respectively, a consensus mutational signature and the mutational catalogue of the examined sample. Hence, \vec{Sᵢ} ∈ [symbol] ₊³² while \vec{M} ∈ N₀³². Further, both vectors have known numerical values either from the consensus mutational signatures (i.e., \vec{Sᵢ}) or from generating the original mutational catalogue of the sample (i.e., \vec{M}). In contrast, Eᵢ corresponds to an unknown scalar reflecting the number of rearrangements contributed by signature \vec{Sᵢ} in the mutational catalogue \vec{M}.

The above equation is universally constrained in regards to the parameter Eᵢ. More specifically, the number of somatic rearrangements contributed by a rearrangement signature in a sample must be nonnegative and it must not exceed the total number of somatic mutations in that sample. Furthermore, the mutations contributed by all signatures in a sample must equal the total number of somatic mutations of that sample. These constraints can be mathematically expressed as 0≤Eᵢ≤∥\vec{Sᵢ}∥₁, i=1 . . . q, and

$${\sum }_{i = 1}^{q} {E}_{i} = {‖ \vec{{S}_{i}} ‖}_{1} .$$

When no prior biological knowledge is available the whole set Q of signatures is used in the determination of Eᵢ, and a filter step is used to move the mutations from the least correlated signatures the ones that best explain the considered sample (signature highly correlated).

Given the catalogue \vec{M} and given all ∥Q^{Q}∥ possible movements between two signatures i and j (i≠j and i, j=1, . . . , Q), the filtering step uses a greedy algorithm to iteratively choose the movement that improves or does not change the cosine similarity between the catalogue \vec{M} and the reconstructed catalogue \vec{M}′=S×\vec{E}′ᵢⱼ. (\vec{E}′ᵢⱼ is the version of the vector \vec{E} obtained by moving the mutations from the signature i to signature j). The filtering step terminates when all the movement between signatures have a negative impact on the cosine similarity.

The filtering step can thus reduce the “noise” in the DNA sample which may initially result in the attribution of a small number of rearrangements to a signature which is not in fact present. The filtering allows such rearrangement to be reassigned to a signature which is more prevalent.

It is then possible to determine whether the sample exhibits one or more of the rearrangement signatures from the known rearrangement signatures from the number of rearrangements which are present in the sample and which are associated with a particular signature. Different thresholds for this determination may be set depending on the context and the desired certainty of the outcome. Generally the threshold will combine the total number of rearrangements detected in the sample (to ensure that the analysis is representative) along with a proportion of the rearrangements which are associated with a particular signature as determined by the above method.

For example, for data obtained from genomes sequenced to 30-40 fold depth, the requirements for detection may be that there are at least 20, preferably at least 50, more preferably at least 100 rearrangements and a signature is deemed to be present if a proportion of at least 10%, preferably at least 20%, more preferably at least 30% of the rearrangements are associated with it. As indicated below, the proportional thresholds may be adjusted depending on the number of other signatures which make up a significant portion of the rearrangements found in the sample (e.g., if 4 signatures are each present with 25% of the rearrangements, then it may be determined that all 4 are present, rather than no signatures at all are present, even if the general requirement for detection is set higher than 25%).

The rearrangement signatures are generally “additive” with respect to each other (i.e. a tumour may be affected by the underlying mutational processes associated with more than one signature and, if this is the case, a sample from that tumour will generally display a higher overall number of rearrangements (being the sum of the separate rearrangements associated with each of the underlying processes), but with the proportion of rearrangements spread over the signatures which are present). As a result, in determining the presence or absence of a particular signature, attention may be paid to the absolute number of rearrangements associated with a particular signature in the sample (as calculated by the method above). Such alternative requirements for detection can better account for the situation where multiple signatures are present. Under this approach, a signature may be determined to be present if at least 10 and preferably at least 20 rearrangements are associated with it.

Method of Detection of Base Substitution Signatures in Single Genomes

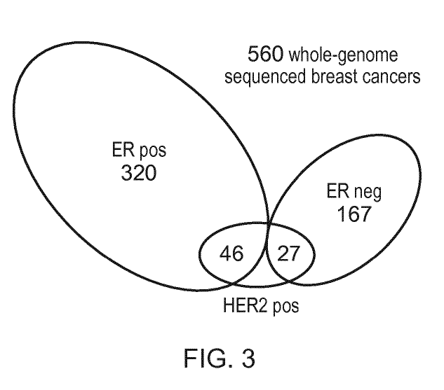

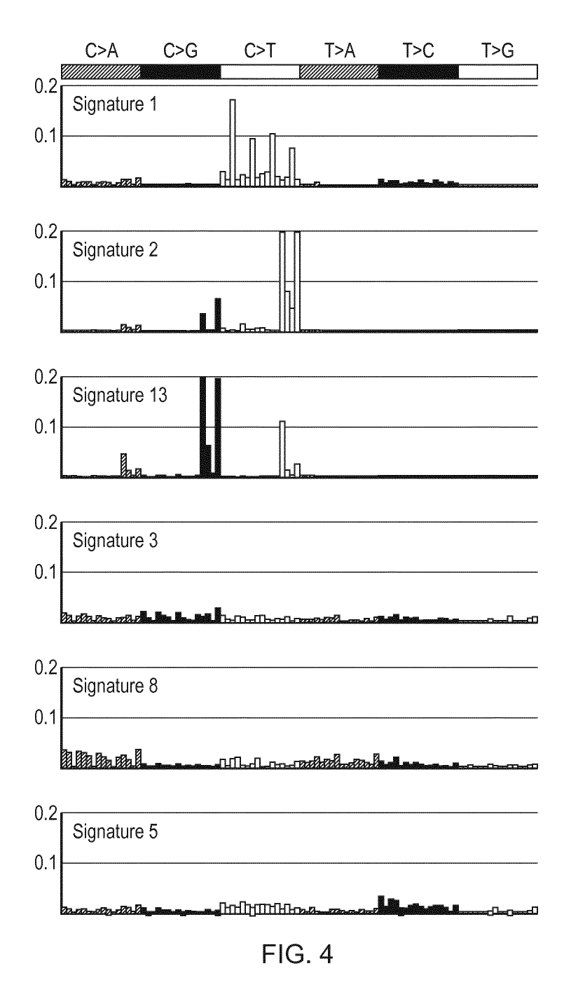

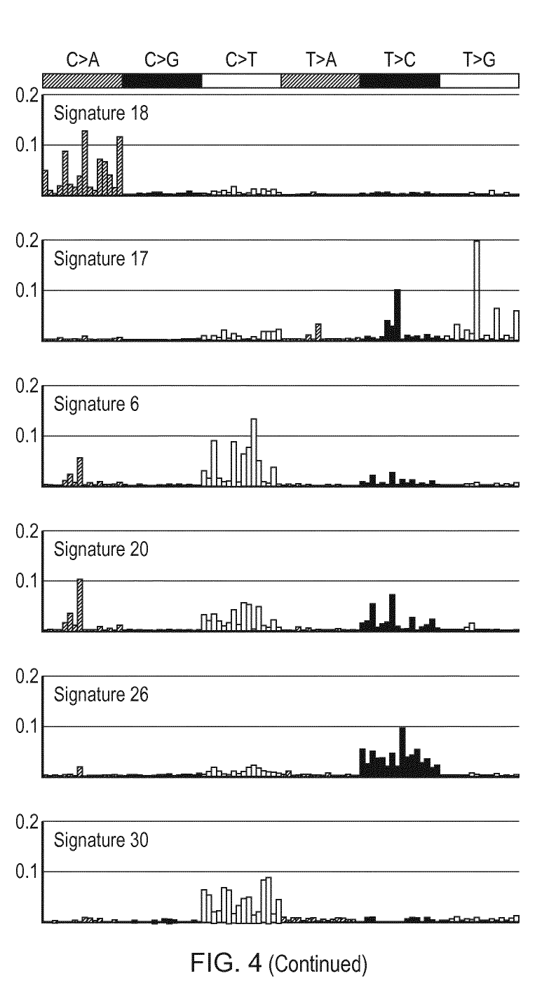

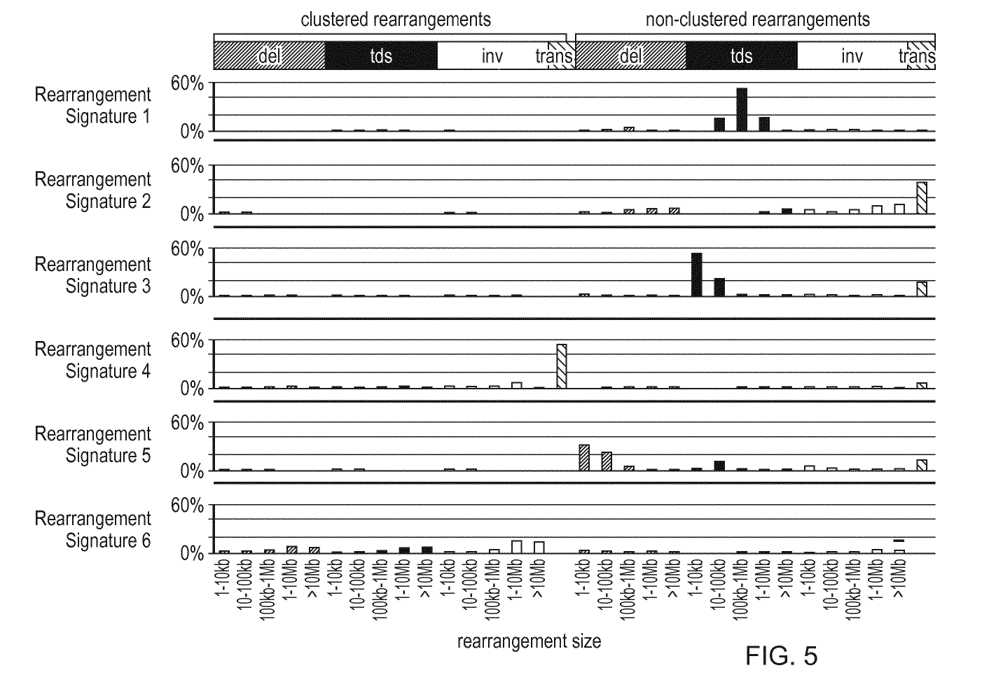

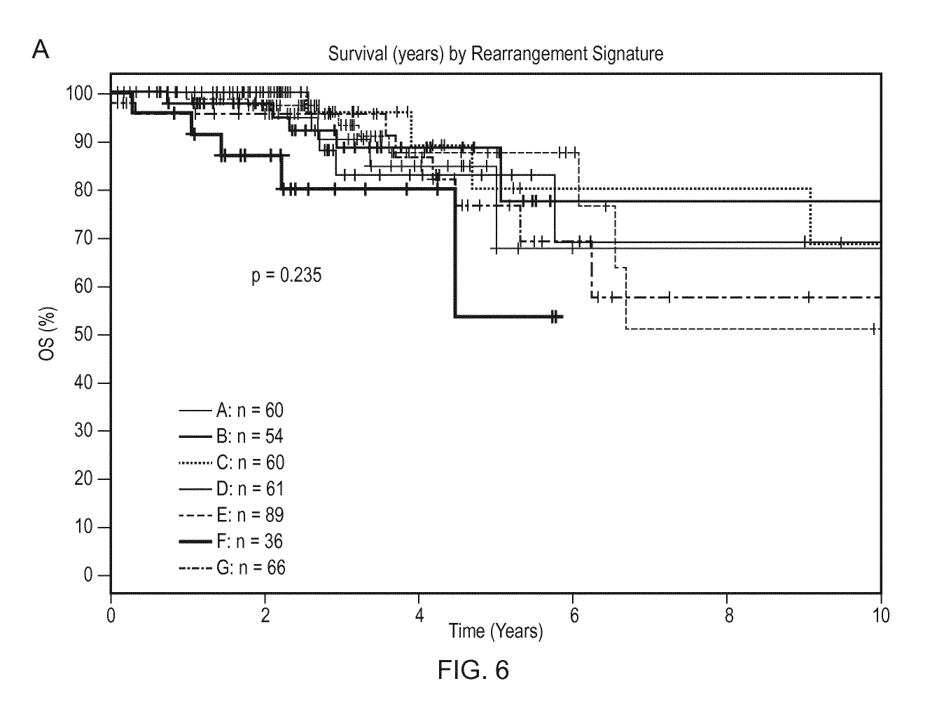

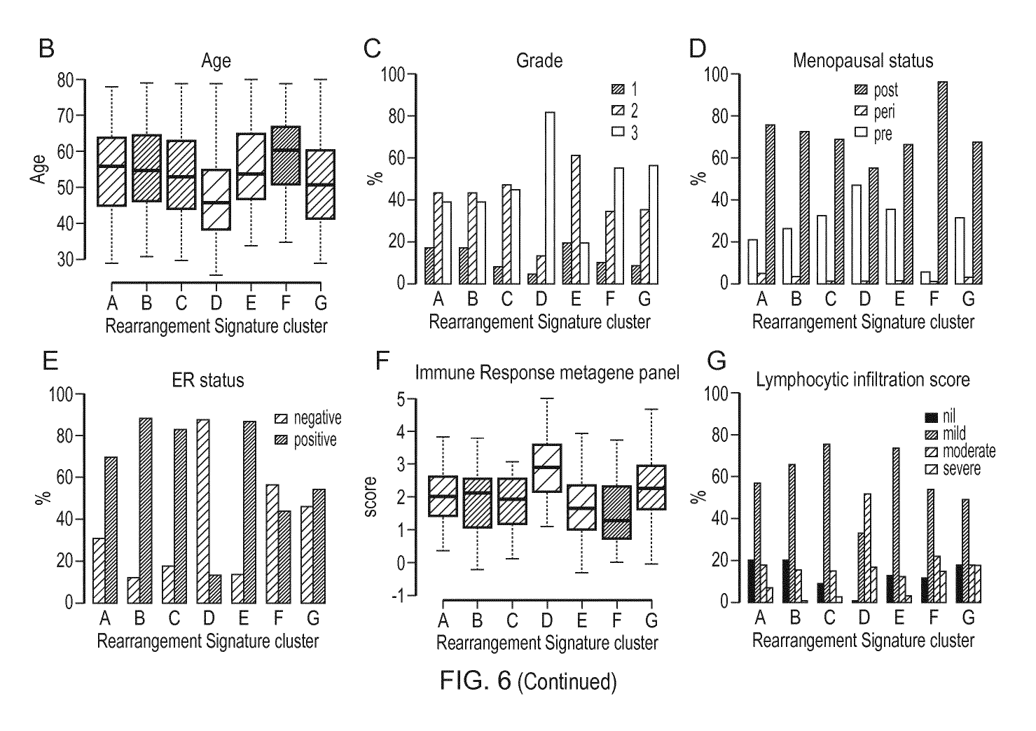

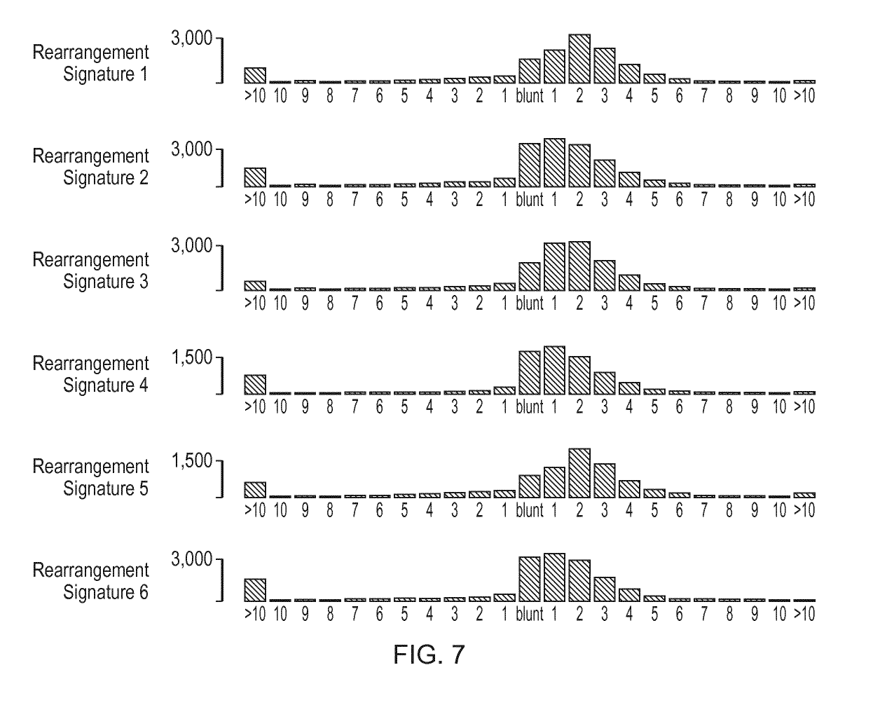

In embodiments of the present invention, detection of a mutational signature in the DNA of a single patient is performed. In these embodiments, this detection is performed by a computer-implemented method or tool that examines a list of somatic mutations generated by targeted, whole-exome, or whole-genome, sequencing of DNA samples obtained from a patient suspected of having cancer. The steps of this method are illustrated schematically in FIG. 3.

The list of somatic mutations for these embodiments can be provided in variety of different formats (including, VCF, MAF, etc.) but at the very minimum needs to contain the following information for each somatic mutation: genome assembly version, chromosome name, start position on the chromosome, end position on the chromosome, reference base(s), mutated base(s).

In broad terms, after loading the list of somatic mutations from the DNA sample (S101) the tool firstly filters out any known germline and/or artifactual somatic mutations (S102), then generates the mutational catalogue of the sample based on single base mutations (S103), evaluates the contributions of known consensus mutational signatures to this sample (S104) and finally determines the set of signatures of mutational processes, and their respective contributions, that are operative in the sample (S105).

By default, the patterns of the consensus mutational signatures are taken from the census website of consensus mutational signatures (http://cancer.sanger.ac.uk/cosmic/signatures) but these patterns of mutational signatures could be also user provided and the method is not limited to known signatures and can be readily applied to new or modified signatures which are discovered in the future.

Filtering Initial Data

Prior to analysing the data, the input list of somatic mutations is extensively filtered to remove any residual germline mutations as well as technology specific sequencing artefacts.

Germline polymorphisms are filtered out from the lists of reported somatic mutations using the complete list of germline mutations from dbSNP (22), 1000 genomes project (23), NHLBI GO Exome Sequencing Project (24) and 69 Complete Genomics panel (http://www.completegenomics.com/public-data/69-Genomes/).

Technology specific sequencing artefacts are filtered out by using panels of BAM files of unmatched normal human tissues containing 300 normal whole-genomes and 570 normal whole-exomes. Any somatic mutation present in at least two well-mapping reads in at least two normal BAM files is discarded. The remaining somatic mutations are used to construct the mutational catalogue of the examined sample.

In specific embodiments of this method, the above filtering is performed by scripts written in Perl.

Generating the Mutational Catalogue for a Sample

The list of remaining (i.e., post-filtered) somatic mutations is used to generate the mutational catalogue of a sample. This mutational catalogue encompasses the six types of somatic substitutions (C:G>A:T, C:G>G:C, C:G>T:A, T:A>A:T, T:A>C:G, and T:A>G:C) and the bases immediately 5′ and 3′ of the somatic mutation, generating 96 possible mutation types (6 types of substitution×4 types of 5′ bases×4 types of 3′ bases).

Thus, each somatic mutation is examined using its genomic position and its immediate 5′ and 3′ bases. The number of somatic mutations and their trinucleotide context are counted based on the pyrimidine base of the mutation.

For example, for human genome build GRCh37, a G:C>A:T mutation on chromosome 9 at position 134147737 will be recorded at CpCpT>CpTpT (mutated base underline and in pyrimidine context). These numbers are aggregated across all somatic mutations left after filtering and they constitute the mutational catalogue of the examined sample.

In specific embodiments of this method, scripts written in Perl, and using the ENSEMBL Core APIs, are used to perform the generation of a mutational catalogue as described above.

In summary, the generation of a mutational catalogue will convert the post-filtered list of somatic mutations into a non-negative vector \vec{M}, where \vec{M} ∈ N₀⁹⁶.

Evaluating the Numbers of Somatic Mutations Attributed to Mutational Signatures in the Mutational Catalogue of the Examined Sample

Calculating the contributions of all mutational signatures is performed by estimating the number of mutations associated to the consensus patterns of the signatures of all operative mutational processes in the sample.

More specifically, all consensus mutational signatures are examined as a set P containing s vectors

$$P = \{ [ \begin{matrix}{p}_{1}^{1} \\ ⋮ \\ {p}_{1}^{96}\end{matrix} ] , [ \begin{matrix}{p}_{2}^{1} \\ ⋮ \\ {p}_{2}^{96}\end{matrix} ] … [ \begin{matrix}{p}_{s - 1}^{1} \\ ⋮ \\ {p}_{s - 1}^{96}\end{matrix} ] , [ \begin{matrix}{p}_{s}^{1} \\ ⋮ \\ {p}_{s}^{96}\end{matrix} ] \} ,$$

where each of the vectors is a discrete probability density function reflecting a consensus mutational signature (by way of example, the vector for signature 3 would be as set out in “Probability” column of Table 3). Here, s refers to the number of known consensus mutational signatures and the 96 nonnegative components of each vector correspond to the number of mutation types (i.e., somatic substitutions and their immediate sequencing context) of these consensus mutational signatures.

The contributions of all consensus mutational signatures are estimated independently for the mutational catalogue of the examined sample. The estimation algorithm consists of finding the minimum of the Frobenius norm of a constrained linear function (see below for constraints) for a set of vectors S_{1 . . . q}, q≤s, belonging to the subset Q, where Q ⊆ P (P is the hitherto mentioned set encompassing all known consensus mutational signatures):

$$min {‖ \vec{M} - {\sum }_{i = 1}^{q} ( \vec{{S}_{i}} \times {E}_{i} ) ‖}_{2}^{F}$$

The subset Q is determined based on prior biological knowledge. This biological knowledge is founded on known characteristics of consensus mutational signatures or on knowledge of the examined sample.

In principle, general biological knowledge about consensus mutational signatures and the cancer types in which they are found is provided at the website: http://cancer.sanger.ac.uk/cosmic/signatures. For example, for any neuroblastoma sample, Q will contain only consensus signatures 1, 5 and 18 since (currently) these are the only known signatures of mutational processes operative in neuroblastoma (see http://cancer.sangerac.uk/cosmic/signatures).

In equation (1), \vec{Sᵢ} and \vec{M} represent vectors with 96 nonnegative components (corresponding to the six somatic substitutions and their immediate sequencing context) reflecting, respectively, a consensus mutational signature and the mutational catalogue of the examined sample. Hence, \vec{Sᵢ} ∈ [symbol] ₊⁹⁶ while \vec{M} ∈ N₀⁹⁶. Further, both vectors have known numerical values either from the census website of consensus mutational signatures (i.e., \vec{Sᵢ}) or from generating the original mutational catalogue of the sample (i.e., \vec{M}). In contrast, Eᵢ corresponds to an unknown scalar reflecting the number of mutations contributed by signature \vec{Sᵢ} in the mutational catalogue \vec{M}.

Minimization of equation (1) is performed under several biologically meaningful linear constraints. The set of vectors in the examined set Q is constrained based on previously identified biological features of the consensus mutational signatures. This can be done computationally by coding the biological conditions into the minimization process.

For example, consensus signature 6 causes high levels of small insertions and/or deletions (indels) at mono/polynucleotide repeats. Thus, this mutational signature will be excluded from the set Q when the mutational catalogue of an examined sample has only a few such indels.

Similarly, there are signatures associated with other types of indels, transcriptional strand bias, dinucleotide mutations, hyper mutator phenotypes, etc. and these signatures are included in the set Q only when the sample in question exhibits one or more of these features. Lists of features associated with mutational signatures can be found at the census website of consensus mutational signatures (http://cancersangerac.uk/cosmic/signatures).

Note that when there is lack of any prior biological knowledge, the complete set of consensus mutational signatures P is used for this analysis.

In addition to biologically meaningful constraints to the set Q, equation (1) is universally constrained in regards to the parameter Eᵢ. More specifically, the number of somatic mutations contributed by a mutational signature in a sample must be nonnegative and it must not exceed the total number of somatic mutations in that sample. Furthermore, the mutations contributed by all signatures in a sample must equal the total number of somatic mutations of that sample. These constraints can be mathematically expressed as 0≤Eᵢ≤∥\vec{Sᵢ}₁, i=1 . . . q, and

$${\sum }_{i = 1}^{q} {E}_{i} = {‖ \vec{{S}_{i}} ‖}_{1} .$$

Numerically, the minimization equation (1) can be examined as finding the minimum of a finite constrained nonlinear multivariable function. This function can be effectively minimized using either the sequential quadratic programming algorithm or the interior-point algorithm. In embodiments of this method, the constrained minimization module is implemented in MATLAB using the fmincon function from the Optimization toolbox.

The minimization procedure results in assigning a number of somatic mutations to each of the examined consensus mutational signatures. These numbers of somatic mutations can be converted to a number of somatic mutations per sequenced megabase by dividing them by the number of sequenced megabases for the sample. Signatures with a contribution less than or equal to 0.01 mutations per sequenced megabase are considered to not be present in the sample, signatures with a contribution higher than 0.01 mutations per sequenced megabase but less than or equal to 0.10 mutations per sequenced megabase are considered to be weakly present in the sample, signatures with a contribution higher than 0.10 mutations per sequenced megabase but less than or equal to 0.35 mutations per sequenced megabase are considered to be present in the sample, and signatures with a contribution higher than 0.35 mutations per sequenced megabase are considered to be strongly present in the sample.

The systems and methods of the above embodiments may be implemented in a computer system (in particular in computer hardware or in computer software) in addition to the structural components and user interactions described.

The term “computer system” includes the hardware, software and data storage devices for embodying a system or carrying out a method according to the above described embodiments. For example, a computer system may comprise a central processing unit (CPU), input means, output means and data storage. Preferably the computer system has a monitor to provide a visual output display (for example in the design of the business process). The data storage may comprise RAM, disk drives or other computer readable media. The computer system may include a plurality of computing devices connected by a network and able to communicate with each other over that network.

The methods of the above embodiments may be provided as computer programs or as computer program products or computer readable media carrying a computer program which is arranged, when run on a computer, to perform the method(s) described above.

The term “computer readable media” includes, without limitation, any non-transitory medium or media which can be read and accessed directly by a computer or computer system. The media can include, but are not limited to, magnetic storage media such as floppy discs, hard disc storage media and magnetic tape; optical storage media such as optical discs or CD-ROMs; electrical storage media such as memory, including RAM, ROM and flash memory; and hybrids and combinations of the above such as magnetic/optical storage media.

The methods of the above embodiments may be provided as computer programs or as computer program products or computer readable media carrying a computer program which is arranged, when run on a computer, to perform the method(s) described above.

The term “computer readable media” includes, without limitation, any non-transitory medium or media which can be read and accessed directly by a computer or computer system. The media can include, but are not limited to, magnetic storage media such as floppy discs, hard disc storage media and magnetic tape; optical storage media such as optical discs or CD-ROMs; electrical storage media such as memory, including RAM, ROM and flash memory; and hybrids and combinations of the above such as magnetic/optical storage media.

### REFERENCES

1 Ford, D. et al. Genetic heterogeneity and penetrance analysis of the BRCA1 and BRCA2 genes in breast cancer families. The Breast Cancer Linkage Consortium. American journal of human genetics 62, 676-689 (1998).
2 King, M. C., Marks, J. H., Mandell, J. B. & New York Breast Cancer Study, G. Breast and ovarian cancer risks due to inherited mutations in BRCA1 and BRCA2. Science 302, 643-646, doi:10.1126/science.1088759 (2003).
3 Risch, H. A. et al. Prevalence and penetrance of germline BRCA1 and BRCA2 mutations in a population series of 649 women with ovarian cancer. American journal of human genetics 68, 700-710, doi:10.1086/318787 (2001).
4 Greer, J. B. & Whitcomb, D. C. Role of BRCA1 and BRCA2 mutations in pancreatic cancer. Gut 56, 601-605, doi:10.1136/gut.2006.101220 (2007).
5 Alexandrov, L. B. et al. Signatures of mutational processes in human cancer. Nature 500, 415-421, doi:10.1038/nature12477 (2013). REF 24 from COMPENDIUM
7 Waddell, N. et al. Whole genomes redefine the mutational landscape of pancreatic cancer. Nature 518, 495-501, doi:10.1038/nature14169 (2015).
8 Merajver, S. D. et al. Somatic mutations in the BRCA1 gene in sporadic ovarian tumours. Nature genetics 9, 439-443, doi:10.1038/ng0495-439 (1995).
9 Miki, Y., Katagiri, T., Kasumi, F., Yoshimoto, T. & Nakamura, Y. Mutation analysis in the BRCA2 gene in primary breast cancers. Nature genetics 13, 245-247, doi:10.1038/ng0696-245 (1996).
9 Jackson, S. P. Sensing and repairing DNA double-strand breaks. Carcinogenesis 23, 687-696 (2002).
10 Nik-Zainal, S. et al. Mutational processes molding the genomes of 21 breast cancers. Cell 149, 979-993, doi:10.1016/j.ce11.2012.04.024 (2012).
11 Walsh, T. et al. Spectrum of mutations in BRCA1, BRCA2, CHEK2, and TP53 in families at high risk of breast cancer. Jama 295, 1379-1388, doi:10.1001/jama.295.12.1379 (2006).
12 Stratton, M. R., Campbell, P. J. & Futreal, P. A. The cancer genome. Nature 458, 719-724, doi:10.1038/nature07943 (2009).
13 Nik-Zainal, S. et al. The life history of 21 breast cancers. Cell 149, 994-1007, doi:10.1016/j.ce11.2012.04.023 (2012).
14 Hicks, J. et al. Novel patterns of genome rearrangement and their association with survival in breast cancer. Genome research 16, 1465-1479, doi:10.1101/gr.5460106 (2006).
15 Bergamaschi, A. et al. Extracellular matrix signature identifies breast cancer subgroups with different clinical outcome. The Journal of pathology 214, 357-367, doi:10.1002/path.2278 (2008).
16 Ching, H. C., Naidu, R., Seong, M. K., Har, Y. C. & Taib, N. A. Integrated analysis of copy number and loss of heterozygosity in primary breast carcinomas using high-density SNP array. International journal of oncology 39, 621-633, doi:10.3892/ijo.2011.1081 (2011).
17 Fang, M. et al. Genomic differences between estrogen receptor (ER)-positive and ER-negative human breast carcinoma identified by single nucleotide polymorphism array comparative genome hybridization analysis. Cancer 117, 2024-2034, doi:10.1002/cncr.25770 (2011).
18 Curtis, C. et al. The genomic and transcriptomic architecture of 2,000 breast tumours reveals novel subgroups. Nature 486, 346-352, doi:10.1038/nature10983 (2012).
19 Pleasance, E. D. et al. A comprehensive catalogue of somatic mutations from a human cancer genome. Nature 463, 191-196, doi:10.1038/nature08658 (2010).
20 Pleasance, E. D. et al. A small-cell lung cancer genome with complex signatures of tobacco exposure. Nature 463, 184-190, doi:10.1038/nature08629 (2010).
21 Banerji, S. et al. Sequence analysis of mutations and translocations across breast cancer subtypes. Nature 486, 405-409, doi:10.1038/nature11154 (2012).
22 Ellis, M. J. et al. Whole-genome analysis informs breast cancer response to aromatase inhibition. Nature 486, 353-360, doi:10.1038/nature11143 (2012).
23 Shah, S. P. et al. The clonal and mutational evolution spectrum of primary triple-negative breast cancers. Nature 486, 395-399, doi:10.1038/nature10933 (2012).
24 Stephens, P. J. et al. The landscape of cancer genes and mutational processes in breast cancer. Nature 486, 400-404, doi:10.1038/nature11017 (2012).
25 West, J. A. et al. The long noncoding RNAs NEAT1 and MALAT1 bind active chromatin sites. Molecular cell 55, 791-802, doi:10.1016/j.molce1.2014.07.012 (2014).
26 Huang, F. W. et al. Highly recurrent TERT promoter mutations in human melanoma. Science 339, 957-959, doi:10.1126/science.1229259 (2013).
27 Vinagre, J. et al. Frequency of TERT promoter mutations in human cancers. Nature communications 4, 2185, doi:10.1038/ncomms3185 (2013).
28 Alexandrov, L. B., Nik-Zainal, S., Wedge, D. C., Campbell, P. J. & Stratton, M. R.

Deciphering signatures of mutational processes operative in human cancer. Cell reports 3, 246-259, doi:10.1016/j.celrep.2012.12.008 (2013).
29 Kalyana-Sundaram, S. et al. Gene fusions associated with recurrent amplicons represent a class of passenger aberrations in breast cancer. Neoplasia 14, 702-708 (2012).
30 Helleday, T., Eshtad, S. & Nik-Zainal, S. Mechanisms underlying mutational signatures in human cancers. Nature reviews. Genetics 15, 585-598, doi:10.1038/nrg3729 (2014).
31 Birkbak, N. J. et al. Telomeric allelic imbalance indicates defective DNA repair and sensitivity to DNA-damaging agents. Cancer discovery 2, 366-375, doi:10.1158/2159-8290.CD-11-0206 (2012).
32 Abkevich, V. et al. Patterns of genomic loss of heterozygosity predict homologous recombination repair defects in epithelial ovarian cancer. British journal of cancer 107, 1776-1782, doi:10.1038/bjc.2012.451 (2012).
33 Popova, T. et al. Ploidy and large-scale genomic instability consistently identify basal-like breast carcinomas with BRCA1/2 inactivation. Cancer research 72, 5454-5462, doi:10.1158/0008-5472.CAN-12-1470 (2012).
34 Kozarewa, I. et al. Amplification-free Illumina sequencing-library preparation facilitates improved mapping and assembly of (G+C)-biased genomes. Nature methods 6, 291-295, doi:10.1038/nmeth.1311 (2009).
35 Li, H. & Durbin, R. Fast and accurate short read alignment with Burrows-Wheeler transform. Bioinformatics 25, 1754-1760, doi:10.1093/bioinformatics/btp324 (2009).
36 Ye, K., Schulz, M. H., Long, Q., Apweiler, R. & Ning, Z. Pindel: a pattern growth approach to detect break points of large deletions and medium sized insertions from paired-end short reads. Bioinformatics 25, 2865-2871, doi:10.1093/bioinformatics/btp394 (2009).
37 Zerbino, D. R. & Birney, E. Velvet: algorithms for de novo short read assembly using de Bruijn graphs. Genome research 18, 821-829, doi:10.1101/gr.074492.107 (2008).
38 Van Loo, P. et al. Allele-specific copy number analysis of tumors. Proceedings of the National Academy of Sciences of the United States of America 107, 16910-16915, doi:10.1073/pnas.1009843107 (2010).

All of the above references are hereby incorporated by reference

TABLE 1

| | | | Probability | | | | | |
|---|---|---|---|---|---|---|---|---|
| Type | Class | Size | Signature 1 | Signature 2 | Signature 3 | Signature 4 | Signature 5 | Signature 6 |
| clustered | deletion | 1-10 kb | 0% | 0% | 0% | 1% | 0% | 1% |
| clustered | deletion | 10-100 kb | 0% | 0% | 0% | 1% | 0% | 1% |
| clustered | deletion | 100 kb-1 Mb | 0% | 0% | 0% | 2% | 0% | 3% |
| clustered | deletion | 1 Mb-10 Mb | 0% | 0% | 0% | 3% | 0% | 7% |
| clustered | deletion | >10 Mb | 0% | 0% | 0% | 1% | 0% | 7% |
| clustered | tandem | 1-10 kb | 0% | 0% | 0% | 0% | 0% | 0% |
| | duplication | | | | | | | |
| clustered | tandem | 10-100 kb | 0% | 0% | 0% | 1% | 0% | 1% |
| | duplication | | | | | | | |
| clustered | tandem | 100 kb-1 Mb | 1% | 0% | 0% | 1% | 0% | 3% |
| | duplication | | | | | | | |
| clustered | tandem | 1 Mb-10 Mb | 0% | 0% | 0% | 3% | 0% | 7% |
| | duplication | | | | | | | |
| clustered | tandem | >10 Mb | 0% | 0% | 0% | 1% | 0% | 7% |
| | duplication | | | | | | | |
| clustered | inversion | 1-10 kb | 0% | 0% | 0% | 3% | 0% | 2% |
| clustered | inversion | 10-100 kb | 0% | 0% | 0% | 2% | 0% | 2% |
| clustered | inversion | 100 kb-1 Mb | 0% | 0% | 0% | 3% | 0% | 5% |
| clustered | inversion | 1 Mb-10 Mb | 0% | 0% | 0% | 6% | 0% | 15% |
| clustered | inversion | >10 Mb | 0% | 0% | 0% | 2% | 0% | 14% |
| clustered | translocation | | 0% | 0% | 0% | 56% | 0% | 0% |
| non-clustered | deletion | 1-10 kb | 0% | 2% | 2% | 0% | 32% | 3% |
| non-clustered | deletion | 10-100 kb | 1% | 1% | 0% | 0% | 22% | 2% |
| non-clustered | deletion | 100 kb-1 Mb | 4% | 5% | 0% | 0% | 5% | 2% |
| non-clustered | deletion | 1 Mb-10 Mb | 1% | 6% | 0% | 1% | 1% | 2% |
| non-clustered | deletion | >10 Mb | 0% | 6% | 1% | 0% | 1% | 2% |
| non-clustered | tandem | 1-10 kb | 0% | 0% | 53% | 0% | 1% | 0% |
| | duplication | | | | | | | |
| non-clustered | tandem | 10-100 kb | 16% | 0% | 22% | 0% | 12% | 0% |
| | duplication | | | | | | | |
| non-clustered | tandem | 100 kb-1 Mb | 54% | 0% | 1% | 0% | 1% | 0% |
| | duplication | | | | | | | |
| non-clustered | tandem | 1 Mb-10 Mb | 17% | 2% | 0% | 1% | 0% | 1% |
| | duplication | | | | | | | |
| non-clustered | tandem | >10 Mb | 0% | 5% | 1% | 0% | 1% | 1% |
| | duplication | | | | | | | |
| non-clustered | inversion | 1-10 kb | 1% | 5% | 1% | 1% | 5% | 1% |
| non-clustered | inversion | 10-100 kb | 2% | 2% | 0% | 0% | 3% | 1% |
| non-clustered | inversion | 100 kb-1 Mb | 2% | 4% | 0% | 0% | 0% | 1% |
| non-clustered | inversion | 1 Mb-10 Mb | 0% | 10% | 0% | 1% | 0% | 4% |
| non-clustered | inversion | >10 Mb | 1% | 12% | 1% | 0% | 2% | 3% |
| non-clustered | translocation | | 1% | 39% | 16% | 7% | 13% | 1% |

TABLE 2

| | Sequence | Signature | Signature |
|---|---|---|---|
| Substitution | Context | 26 | 30 |
| C > A | ACA | 0.2040% | 0.0000% |
| C > A | ACC | 0.1487% | 0.0000% |
| C > A | ACG | 0.0284% | 0.1967% |
| C > A | ACT | 0.0598% | 0.0000% |
| C > A | CCA | 0.3706% | 0.0000% |
| C > A | CCC | 0.3981% | 0.0000% |
| C > A | CCG | 0.0812% | 0.2262% |
| C > A | CCT | 1.9038% | 0.0000% |
| C > A | GCA | 0.1375% | 0.8853% |
| C > A | GCC | 0.1962% | 0.9345% |
| C > A | GCG | 0.0013% | 0.0885% |
| C > A | GCT | 0.1935% | 0.8165% |
| C > A | TCA | 0.2680% | 0.0000% |
| C > A | TCC | 0.2032% | 0.0000% |
| C > A | TCG | 0.0265% | 0.1672% |
| C > A | TCT | 0.3017% | 0.0000% |
| C > G | ACA | 0.1273% | 0.0000% |
| C > G | ACC | 0.1528% | 0.0000% |
| C > G | ACG | 0.0307% | 0.4820% |
| C > G | ACT | 0.2498% | 0.0000% |
| C > G | CCA | 0.1279% | 0.0000% |
| C > G | CCC | 0.1215% | 0.0000% |
| C > G | CCG | 0.0208% | 0.3246% |
| C > G | CCT | 0.2297% | 0.0000% |
| C > G | GCA | 0.1321% | 0.7378% |
| C > G | GCC | 0.1846% | 0.6591% |
| C > G | GCG | 0.0205% | 0.1574% |
| C > G | GCT | 0.1226% | 0.0000% |
| C > G | TCA | 0.4202% | 0.0000% |
| C > G | TCC | 0.2808% | 0.0000% |
| C > G | TCG | 0.0000% | 0.1967% |
| C > G | TCT | 0.8019% | 0.0000% |
| C > T | ACA | 0.5907% | 6.5119% |
| C > T | ACC | 1.0626% | 5.4397% |
| C > T | ACG | 1.9930% | 2.0460% |
| C > T | ACT | 1.1335% | 2.1936% |
| C > T | CCA | 0.6594% | 6.9447% |
| C > T | CCC | 0.6511% | 6.3840% |
| C > T | CCG | 1.1905% | 1.7313% |
| C > T | CCT | 0.6239% | 3.4232% |
| C > T | GCA | 0.9607% | 4.8593% |
| C > T | GCC | 1.9507% | 4.9479% |
| C > T | GCG | 2.2503% | 1.5739% |
| C > T | GCT | 1.7307% | 1.8887% |
| C > T | TCA | 1.1303% | 8.4989% |
| C > T | TCC | 1.0808% | 9.0301% |
| C > T | TCG | 1.0364% | 1.5149% |
| C > T | TCT | 0.7249% | 4.5544% |
| T > A | ATA | 0.4459% | 0.7574% |
| T > A | ATC | 1.2822% | 0.3738% |
| T > A | ATG | 0.1172% | 0.6591% |
| T > A | ATT | 0.3993% | 0.9345% |
| T > A | CTA | 0.3561% | 0.5312% |
| T > A | CTC | 0.3902% | 0.6787% |
| T > A | CTG | 0.2390% | 0.8263% |
| T > A | CTT | 0.1636% | 0.0000% |
| T > A | GTA | 0.2243% | 0.3738% |
| T > A | GTC | 0.5207% | 0.3345% |
| T > A | GTG | 0.1358% | 0.5017% |
| T > A | GTT | 0.2513% | 0.6394% |
| T > A | TTA | 0.0628% | 0.7673% |
| T > A | TTC | 0.5074% | 0.6492% |
| T > A | TTG | 0.0020% | 0.3640% |
| T > A | TTT | 0.1236% | 0.0000% |
| T > C | ATA | 5.5029% | 0.0000% |
| T > C | ATC | 2.7595% | 0.8755% |
| T > C | ATG | 5.1791% | 0.9050% |
| T > C | ATT | 3.9072% | 0.0000% |
| T > C | CTA | 3.7889% | 0.0000% |
| T > C | CTC | 2.1741% | 0.0000% |
| T > C | CTG | 4.7240% | 0.0000% |
| T > C | CTT | 2.0741% | 0.0000% |
| T > C | GTA | 9.8053% | 0.0000% |
| T > C | GTC | 4.0226% | 0.6591% |
| T > C | GTG | 4.4621% | 0.7869% |
| T > C | GTT | 5.5528% | 0.8460% |
| T > C | TTA | 2.8790% | 0.0000% |
| T > C | TTC | 3.6639% | 0.9148% |
| T > C | TTG | 1.9144% | 0.6000% |
| T > C | TTT | 2.3072% | 0.0000% |
| T > G | ATA | 0.0081% | 0.5312% |
| T > G | ATC | 0.0163% | 0.2459% |
| T > G | ATG | 0.1255% | 0.6296% |
| T > G | ATT | 0.1850% | 0.9542% |
| T > G | CTA | 0.0095% | 0.4623% |
| T > G | CTC | 0.3919% | 0.6000% |
| T > G | CTG | 0.5497% | 0.7378% |
| T > G | CTT | 0.6789% | 0.9148% |
| T > G | GTA | 0.0037% | 0.3246% |
| T > G | GTC | 0.2461% | 0.1869% |
| T > G | GTG | 0.0817% | 0.3345% |
| T > G | GTT | 0.7834% | 0.5607% |
| T > G | TTA | 0.0009% | 0.8656% |
| T > G | TTC | 0.2719% | 0.4328% |
| T > G | TTG | 0.1369% | 0.8263% |
| T > G | TTT | 0.2568% | 0.0000% |

1. A method of predicting whether a patient with cancer is likely to respond to a PARP inhibitor or a platinum-based drug, the method comprising determining the presence or absence of one or more of rearrangement signatures 1, 3 and/or 5 in a DNA sample obtained from said patient, wherein rearrangement signatures 1, 3 and 5 are defined in Table 1 and a DNA sample is considered to show the presence of a rearrangement signature if the number or proportion of rearrangements in its rearrangement catalogue which are determined to be associated with one or more of said rearrangement signatures each or in combination exceeds a predetermined threshold, wherein if one of said rearrangement signatures is present in the sample, the patient is likely to respond to a PARP inhibitor or a platinum-based drug.

2. A method of selecting a patient having cancer for treatment with a PARP inhibitor or a platinum-based drug, the method comprising identifying the presence or absence of one or more of rearrangement signatures 1, 3 and/or 5 in a DNA sample obtained from said patient, wherein rearrangement signatures 1, 3 and 5 are defined in Table 1 and a DNA sample is considered to show the presence of a rearrangement signature if the number or proportion of rearrangements in its rearrangement catalogue which are determined to be associated with one or more of said rearrangement signatures each or in combination exceeds a predetermined threshold, and selecting the patient for treatment with a PARP inhibitor or a platinum-based drug if one of said rearrangement signatures is present in the sample.

3. A PARP inhibitor or a platinum-based drug for use in a method of treatment of cancer in a patient having one or more of rearrangement signatures 1, 3 and/or 5, wherein rearrangement signatures 1, 3 and 5 are defined in Table 1 and a DNA sample is considered to show the presence of a rearrangement signature if the number or proportion of rearrangements in its rearrangement catalogue which are determined to be associated with one or more of said rearrangement signatures each or in combination exceeds a predetermined threshold.

4. A method of treating cancer in a patient determined to have one or more of rearrangement signatures 1, 3 and/or 5, wherein rearrangement signatures 1, 3 and 5 are defined in Table 1 and a DNA sample is considered to show the presence of a rearrangement signature if the number or proportion of rearrangements in its rearrangement catalogue which are determined to be associated with one or more of said rearrangement signatures each or in combination exceeds a predetermined threshold, the method comprising the step of administering a PARP inhibitor or a platinum-based drug to said patient.

5. A PARP inhibitor or a platinum-based drug for use in a method of treatment of cancer in a patient, the method comprising:
(i) determining whether one or more of rearrangement signatures 1, 3 and/or 5 is present in a DNA sample obtained from said patient, wherein rearrangement signatures 1, 3 and 5 are defined in Table 1 and a DNA sample is considered to show the presence of a rearrangement signature if the number or proportion of rearrangements in its rearrangement catalogue which are determined to be associated with one or more of said rearrangement signatures each or in combination exceeds a predetermined threshold; and
(ii) administering the PARP inhibitor or a platinum-based drug to a patient if one of said rearrangement signatures is present in said sample.

6. A method of determining the presence of any one of rearrangement signatures 1 to 6 in a DNA sample obtained from a patient, wherein the rearrangement signatures are defined in Table 1 and a DNA sample is considered to show the presence of a particular rearrangement signature if the number or proportion of rearrangements in its rearrangement catalogue which are determined to be associated with that particular rearrangement signature exceeds a predetermined threshold.

7. The method according to any one of claim 1, 2, 4 or 6 wherein the step of determining the presence or absence of a rearrangement signature in the sample includes the steps of:
cataloguing the somatic mutations in said sample to produce a rearrangement catalogue for that sample which classifies identified rearrangement mutations in the sample into a plurality of categories; and
determining the contributions of known rearrangement signatures to said rearrangement catalogue by computing the cosine similarity between the rearrangement mutations in said catalogue and the rearrangement mutational signatures.

8. The method according to claim 7 wherein the method includes the further step of, prior to said step of determining, filtering the mutations in said catalogue to remove one or more of: residual germline mutations; copy number polymorphisms; and known sequencing artefacts.

9. The method according to claim 8 wherein the filtering uses a list of known germline polymorphisms.

10. The method according to claim 8 wherein the filtering uses BAM files of unmatched normal human tissue sequenced by the same process as the DNA sample and discards any somatic mutation which is present in at least two well-mapping reads in at least two of said BAM files.

11. The method according any one claims 7 to 10 wherein the classification of the rearrangement mutations includes identifying mutations as being clustered or non-clustered.

12. The method according to claim 11 wherein mutations are identified as being clustered if they have an average density of rearrangement breakpoints that is at least 10 times greater the whole genome average density of rearrangements for an individual patient's sample.

13. The method according to any one of claims 7 to 12 wherein the classification of the rearrangement mutations includes identifying mutations as one of: tandem duplications, deletions, inversions or translocations.

14. The method according to claim 13 wherein the classification of the rearrangement mutations includes grouping mutations identified as tandem duplications, deletions or inversions by size.

15. The method according to any one of claims 7 to 14 further including the step of determining the number of rearrangements \vec{E}, in the rearrangement catalogue associated with the ith known mutational signature \vec{S}ᵢ, which is proportional to the cosine similarity (\vec{C}ᵢ) between the catalogue of this sample \vec{M} and \vec{S}ᵢ:
$${\vec{C}}_{i} = \frac{{\vec{S}}_{i} \cdot \vec{M}}{‖ {\vec{S}}_{i} ‖ ‖ \vec{M} ‖}$$
$$wherein \text{:}$$
$${E}_{i} = \frac{{\vec{C}}_{i}}{{\sum }_{i = 1}^{q} {\vec{C}}_{i}} {\sum }_{j = 1}^{36} {\vec{M}}^{j}$$
wherein \vec{Sᵢ}, and \vec{M} are equally-sized vectors with nonnegative components being, respectively, the known rearrangement signature and the rearrangement catalogue and q is the number of signatures in said plurality of known rearrangement signatures, and wherein Eᵢ are further constrained by the requirements that 0≤Eᵢ≤∥\vec{Sᵢ}∥₁, i=1 . . . q, and
$${\sum }_{i = 1}^{q} {E}_{i} = {‖ \vec{{S}_{i}} ‖}_{1} .$$

16. The method according to claim 15 wherein the step of determining the number of rearrangements further includes the step of filtering the number of rearrangements determined to be assigned to each signature by reassigning one or more rearrangements from signatures that are less correlated with the catalogue to signatures that are more correlated with the catalogue.

17. The method according to claim 16 wherein the step of filtering uses a greedy algorithm to iteratively find an alternative assignment of rearrangements to signatures that improves or does not change the cosine similarity between the catalogue \vec{M} and the reconstructed catalogue \vec{M}′=S ×\vec{E}′ᵢⱼ, wherein \vec{E}′ᵢⱼ is the version of the vector \vec{E} obtained by moving the mutations from the signature i to signature j, wherein, in each iteration, the effects of all possible movements between signatures are estimated, and the filtering step terminates when all of these possible reassignments have a negative impact on the cosine similarity.

18. A method of detecting mutational signature 26 or mutational signature 30 in a DNA sample, wherein mutational signatures 26 and 30 are defined in Table 2, the method including the steps of: cataloguing the somatic mutations in said sample to produce a mutational catalogue for that sample; determining the contributions of known mutational signatures, including mutational signature 26 or mutational signature 30, to said mutational catalogue by determining a scalar factor for each of a plurality of said known mutational signatures which together minimize a function representing the difference between the mutations in said catalogue and the mutations expected from a combination of said plurality of known mutational signatures scaled by said scalar factors; and if the scalar factor corresponding to mutational signature 26 or mutational signature 30 exceeds a predetermined threshold, identifying said sample as containing corresponding mutational signature 26 or mutational signature 30 respectively.

19. The method according to claim 18 wherein the method includes the further step of, prior to said step of determining, filtering the mutations in said catalogue to remove either residual germline mutations or known sequencing artefacts or both.

20. The method according to claim 19 wherein the filtering uses a list of known germline polymorphisms.

21. The method according to claim 19 or claim 20 wherein the filtering uses BAM files of unmatched normal human tissue sequenced by the same process as the DNA sample and discards any somatic mutation which is present in at least two well-mapping reads in at least two of said BAM files.

22. The method according to any one of claims 18 to 21 further including the step of selecting said plurality of known mutational signatures as a subset of all known mutational signatures.

23. The method according to claim 22 wherein the subset of mutational signatures is selected based on biological knowledge about the DNA sample or the mutational signatures or both.

24. The method according to any one of claims 18 to 23 wherein the step of determining determines the scalars Eᵢ which minimize the Frobenius norm:
$$min {‖ \vec{M} - {\sum }_{i = 1}^{q} ( \vec{{S}_{i}} \times {E}_{i} ) ‖}_{2}^{F}$$
wherein \vec{Sᵢ} and \vec{M} are equally-sized vectors with nonnegative components being, respectively, a consensus mutational signature and the mutational catalogue and q is the number of signatures in said plurality of known mutational signatures, and wherein Eᵢ are further constrained by the requirements that 0≤Eᵢ≤∥\vec{Sᵢ}∥₁, i=1 . . . q, and
$${\sum }_{i = 1}^{q} {E}_{i} = {‖ \vec{{S}_{i}} ‖}_{1} .$$
